(19) United States
(12) Patent Application Publication
(10) Pub. No.: US 2017/0306096 A1
(43) Pub. Date: Oct. 26, 2017

(54) MOISTURE CURABLE COMPOSITIONS

(71) Applicant: Momentive Performance Materials Inc., Waterford, NY (US)

(72) Inventors: Ramesh MUTHUSAMY, Bangalore (IN); Sumi DINKAR, Bangalore (IN); Anantharaman DHANABALAN, Bangalore (IN)

(21) Appl. No.: 15/528,114

(22) PCT Filed: Nov. 20, 2015
(86) PCT No.: PCT/US15/61739

§ 371 Date: May 19, 2017

Related U.S. Application Data
(60) Provisional application No. 62/082,406, filed on Nov. 20, 2014.

(51) Int. Cl. C08G 77/08; C07C 229/24; C08G 77/38; C08K 5/544

(52) U.S. Cl. CPC C08G 77/08; C08K 5/544; C07C 229/24; C08G 77/38

(57) ABSTRACT

The present invention provides moisture-curable compositions comprising an amino ester catalyst as an alternative to organotin catalysts. In particular, the present invention provides a condensation catalyst comprising a secondary amine, a tertiary amine, a substituted amine (e.g., an amino ester compound), or a combination of two or more thereof and optionally one or more aminosilanes or siloxanes. Further, the compositions employing amino esters allow for tuning or adjusting the cure characteristics of the compositions by the addition of other components such as adhesion promoters or acidic compounds, and provides good adhesion and storage stability.

### CROSS-REFERENCE TO RELATED APPLICATIONS

The present application claims priority to and the benefit of U.S. Provisional Application No. 62/082,406, titled “Moisture Curable Compositions,” filed on Nov. 20, 2014, the entire disclosure of which is incorporated herein by reference.

### FIELD

The present invention relates to moisture-curable compositions comprising an amine compound as a catalyst. In particular, the present invention provides curable compositions comprising a secondary amine, a tertiary amine or substituted amine catalyst as an alternative to organotin catalysts. The catalyst may be, for example, a linear or cyclic aliphatic amine, an aromatic amine, a heterocyclic amine, an amino ester, etc.

### BACKGROUND

Polymers having reactive-silyl groups or compositions comprising such polymers can be hydrolyzed and condensed in the presence of water and metal catalysts. Suitable known catalysts for curable compositions include compounds employing metals such as Sn, Ti, Zn, or Ca. Organotin compounds such as, for example, dibutyltin dilaurate (DBTDL) are widely used as condensation cure catalysts to accelerate the moisture-assisted curing of a number of different polyorganosiloxanes and non-silicone polymers having reactive terminal silyl groups such as room temperature vulcanizing (RTV) formulations including RTV-1 and RTV-2 formulations. Environmental regulatory agencies and directives, however, have increased or are expected to increase restrictions on the use of organotin compounds in formulated products. For example, while formulations with greater than 0.5 wt. % dibutyltin presently require labeling as toxic with reproductive 1B classification, dibutyltin-containing formulations are proposed to be completely phased out in consumer applications during the next two to three years.

The use of alternative organotin compounds such as dioctyltin compounds and dimethyltin compounds can only be considered as a short-term remedial plan, as these organotin compounds may also be regulated in the future. It would be beneficial to identify non-tin-based catalysts that accelerate the condensation curing of moisture-curable silicones and non-silicones.

Substitutes for organotin catalysts should exhibit properties similar to organotin compounds in terms of curing, storage, and appearance. Non-tin catalysts would also desirably initiate the condensation reaction of the selected polymers and complete this reaction upon the surface and may be in the bulk in a desired time schedule. There are therefore many proposals for the replacement of organometallic tin compounds with other metal- and non-metal-based compounds. These new catalysts have specific advantages and disadvantages in view of replacing tin compounds perfectly. Therefore, there is still a need to address the weaknesses of possible non-tin compounds as suitable catalysts for condensation cure reactions. The physical properties of uncured and cured compositions also warrant examination, in particular to maintain the ability to adhere onto the surface of several substrates.

Prior replacement catalysts for organotin compounds generally cannot maintain their ability to cure when exposed to humidity or ambient air after storage over months in a sealed cartridge. It is always a specific requirement for moisture-curable compositions to achieve the shortest possible curing times, showing a tack-free surface as well as curing through the complete bulk in thick section for RTV-1 and RTV-2 compositions. Additionally, such compositions should provide a reasonable adhesion after cure onto a variety of substrates. Thus, there is still a need for alternative materials to replace tin as a core catalyst in moisture curable compositions.

### SUMMARY

The present invention provides tin-free, curable compositions comprising silyl-terminated polymers and a catalyst comprising an amine compound chosen from a secondary amine, a tertiary amine or a substituted amine compound. In embodiments, the amine catalyst may be chosen from a linear or cyclic aliphatic amine, an aromatic amine, a heterocyclic amine, an amino ester, or a combination of two or more thereof. In one embodiment, the present invention provides curable compositions employing an amino ester compound as a catalyst in a moisture curable composition.

In one embodiment, the curable composition comprises (A) a polymer having a reactive silicon-containing group, (B) a cross-linker and/or a chain extender, (C) a catalyst comprising a linear or cyclic aliphatic amine, an aromatic amine, a heterocyclic amine, an amino ester compound, or a combination of two of there thereof, (D) optionally an adhesion promoter, (E) optionally a filler, and (F) optionally a cure accelerator, and (G) optionally an auxiliary component.

In one aspect, the invention provides a curable composition exhibiting a relatively short tack-free time, curing through the bulk, as well as long storage stability in the cartridge, i.e., in the absence of humidity. Compounds with an amino ester functionality have been found to exhibit good curing behavior, including good tack free time and/or bulk curing. Use of the amino ester compounds with adhesion promoters may allow for tuning the cure properties of the composition. Thus, the amino ester can be suitable as replacements for organotin catalysts in compositions having a reactive, silyl-terminated polymer that can undergo condensation reactions, such as in RTV-1 and RTV-2 formulations.

Curable compositions using linear or cyclic aliphatic amine, aromatic amine, heterocyclic amine, and/or amino esters may also exhibit certain storage stability of the uncured composition in the cartridge, adhesion onto several surfaces, and a cure rate in a predictable time scheme.

In one aspect, the present invention provides a composition for forming a cured polymer composition comprising: (A) a polymer having at least one reactive silyl group; (B) a crosslinker or chain extender chosen from an alkoxysilane, an alkoxysiloxane, an oximosilane, an oximosiloxane, an enoxysilane, an enoxysiloxane, an aminosilane, an aminosiloxane, a carboxysilane, a carboxysiloxane, an alkylamidosilane, an alkylamidosiloxane, an arylamidosilane, an arylamidosiloxane, an alkoxyaminosilane, an alkoxyaminosiloxane, an alkoxycarbamatosilane, an alkoxycarbamatosiloxane, and combinations of two or more thereof; (C) a catalyst chosen from a secondary amine, a tertiary amine or a substituted amine such as a linear or cyclic aliphatic amine, an aromatic amine, a heterocyclic amine, an amino ester compound, or a combination of two or more thereof; (D) optionally at least one adhesion promoter chosen from a silane or siloxane other than the compounds listed under (B); (E) optionally a filler component; (F) optionally a acidic component; and (G) optionally an auxiliary component comprising an organo-functional silicon compound and or low molecular weight organic polymer and or high boiling solvents.

In one embodiment, the present invention provides a curable composition according to any previous embodiment that is substantially free of tin.

In one embodiment, the present invention provides a curable composition according to any previous embodiment, wherein the catalyst comprises a plurality of amine functional groups.

In one embodiment, the present invention provides a curable composition according to any previous embodiment, wherein the amine compound comprises one or multiple amine functional group of the formula:

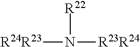

where R²² is independently chosen from hydrogen; a C₁-C₁₅ linear, branched, or cyclic alkyl group; a C₁-C₁₅ linear, branched, or cyclic alkyl group comprising one or more substituents chosen from a halide, N, O or S; a C₆-C₁₀ aryl group; a C₇-C₁₆ linear or branched alkylaryl group; a C₂-C₄ polyalkylene ether; or a linear or branched C₇-C₁₆ heteroaralkyl, heteroalkyl, heterocycloalkyl, or heteroaryl; and where R²³ and R²⁴ are independently chosen from a C₁-C₁₅ linear, branched, or cyclic alkyl group; a C₁-C₁₅ linear, branched, or cyclic alkyl group comprising one or more substituents chosen from a halide, N, O or S; a C₆-C₁₀ aryl group; a C₇-C₁₆ linear or branched alkylaryl group; a C₂-C₄ polyalkylene ether; a linear or branched C₇-C₁₆ heteroaralkyl; heteroalkyl, heterocycloalkyl, heteroaryl, with the proviso that the N atom is bi-substituted with either of R²³, R²⁴, R²³ and R²⁴, or a combined R²³, R²⁴ in the compound.

In one embodiment, the present invention provides a curable composition according to any previous embodiment, wherein the catalyst comprises a secondary amine selected from dialkyl and substituted dialkyl amines, dimethylamine, diisopropylamine, dibutylamine, N-methylbutylamine, N,N-diallyl trimethylenediamine, diamylamine, dihexylamine, dioctylamine, N-ethylcetylamine, didodecylamine, ditetradecylamine, diricinoleylamine, N-isopropylstearylamine, N-isoamylhexylamine, N-ethyloctylamine, dioctadecylamine, their homologs and analogs, or a combination of two or more thereof

In one embodiment, the present invention provides a curable composition according to any previous embodiment, wherein the catalyst comprises a secondary cycloalkylamine selected from dicyclohexylamine, N-methylcyclohexylamine, dicyclopentylamine, N-octylcyclohexylamine, N-octyl-3,5,5-trimethylcyclohexylamine, diallylamine, N-ethylallylamine, N-octylallylamine, dioleylamine, N-isopropylolelyamine, N-methyl-3,3,5-trimethyl-5-cyclohexenylamine, N-amyl-linoleylamine, N-methyl-propargylamine, diphenylamine, their analogs and homologs, or a combination of two or more thereof

In one embodiment, the present invention provides a curable composition according to any previous embodiment, wherein the catalyst comprises a tertiary amine selected from triethylamine, tri-isopropylamine, tributylamine, N-ethyldibutylamine, N-ethyl-N-butylamylamine, N,N-diethyl aniline, triallylamine, N,N-dipropylcyclohexylamine, N,N-dipropyloleyl-amine, trimethylamine, N-octyldiallylamine, N,N-dipropylcyclohexylamine, dimethylaminopropylemine, dimethylaminoethoxypropylamine, pentamethyldiethylylenetriamine, trimethylamine, triethylamine, N-methylmorpholine, N-ethylmorpholine, N,N-dimethylbenzylamine, N,N-dimethylethanolamine, N,N,N′,N′-tetramethyl-1,4-butanediamine, N,N-dimethylpiperazine, bis(2-dimethylaminoethyl)ether, morpholine, N-substituted morpholines, such as N-methyl or N-ethyl morpholine, 4,4′-(oxydi-2,l-ethanediyl)bis, triethylenediamine, pentamethyl diethylene triamine, dimethyl cyclohexyl amine, N-cetyl N,N-dimethyl amine, N-coco-morpholine, N,N-dimethyl aminomethyl N-methyl ethanol amine, N,N,N′-trimethyl-N′-hydroxyethyl bis(aminoethyl)ether, N,N-bis(3-dimethylaminopropyl)N-isopropanolamine, (N,N-dimethyl)amino-ethoxy ethanol, N,N,N′,N′-tetramethyl hexane diamine, N,N-dimorpholinodiethyl ether, N-methyl imidazole, dimethyl aminopropyl dipropanolamine, bis(dimethylaminopropyl)amino-2-propanol, tetramethylamino bis (propylamine), (dimethyl(aminoethoxyethyl))((dimethyl amine)ethyl)ether, tris(dimethylamino propyl) amine, dicyclohexyl methyl amine, bis(N,N-dimethyl-3-aminopropyl) amine, N,N-bis (3-dimethylaminopropyl)-N-isopropanolamine, 1,3-propanediamine, 1,2-ethylene piperidine, methyl-hydroxyethyl piperazine, dimethylaminopropyl-S-triazine, bisdimethylaminopropylurea, their analogs and homologs, or a combination of two or more thereof.

In one embodiment, the present invention provides a curable composition according to any previous embodiment, wherein the catalyst comprises a heterocyclic amine selected from piperidine, pyridine, methylpiperazine, 2,2,4,6-tetramethylpiperidine, 2,2,4,6-tetramethyl-tetrahydropyridine, N-ethyl 2,2,4,6 tetramethylpiperidine, 2-aminopyrimidine, 2- aminopyridine, 2-(dimethylamino)pyridine, 4-(dimethylamino)pyridine, 2-hydroxypyridine, imidazole, 2-ethyl-4-methylimidazole, morpholine, N-methylmorpholine, 2-piperidinemethanol, 2-(2-piperidino)ethanol, piperidone, 1,2-dimethyl-1,4,5,6-tetrahydropyrimidine, aziridine, methoymethyldiphenylamine, nicotine, pentobarbital, methadone, cocaine, and triphenylamine, or a combination of two or more thereof.

In one embodiment, the present invention provides a curable composition according to any previous embodiment, wherein the catalyst comprises a substituted amine is chosen from an amino ester compound.

In one embodiment, the present invention provides a curable composition according to any previous embodiment, wherein the amino ester compound comprises at least one amino ester functional group.

In one embodiment, the present invention provides a curable composition according to any previous embodiment, wherein the amino ester compound comprises 1-10 amino ester functional groups.

In one embodiment, the present invention provides a curable composition according to any previous embodiment, wherein the amino ester compound comprises 1-4 amino ester functional groups.

In one embodiment, the present invention provides a curable composition according to any previous embodiment, wherein the amino ester compound comprises an amino ester functional group of the formula:

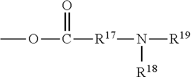

where R¹⁷ is a C₁-C₅ alkyl group, and R¹⁸ and R¹⁹ are independently chosen from hydrogen, a C₁-C₁₀ linear, branched, or cyclic alkyl group, a C₁-C₁₀ linear, branched, or cyclic alkyl group comprising one or more substituents chosen from a halide, N, O or S; a C₆-C₁₀ aryl group; a C₇-C₁₆ alkylaryl group; a C₇-C₁₆ arylalkyl group; a C₂-C₄ polyalkylene ether; a substituted silicon, a substituted siloxane, or a combination of two or more thereof.

In one embodiment, the present invention provides a curable composition according to any previous embodiment, wherein the amino ester has a molecular weight of from about 50 g/mol to about 10000 g/mol.

In one embodiment, the present invention provides a curable composition according to any previous embodiment, wherein the amino ester has a pKa of from about 3.0 to about 9.0.

In one embodiment, the present invention provides a curable composition according to any previous embodiment, wherein the curable composition comprises from about 0.0001 to about 10 parts per weight of catalyst (C) per 100 parts per weight of the polymer (A). In another embodiment, the curable composition comprises from about 0.005 to about 0.05 wt. pt. of catalyst (C) per 100 parts of the polymer (A). In another embodiment, the catalyst component (C) is present in an amount of from about 0.15 to about 2.0 wt. pt. based on 100 parts of the polymer component (A).

In one embodiment, the present invention provides a curable composition according to any previous embodiment, wherein the polymer (A) has the formula: [R¹ₐR²₃₋ₐSi-Z-]-X-Z-SiR¹ₐR²₃₋ₐ. In another embodiment, X is chosen from a polyurethane; a polyester; a polyether; a polycarbonate; a polyolefin; a polyesterether; and a polyorganosiloxane having units of R₃SiO_{1/2}, R₂SiO, RSiO_{3/2}, and/or SiO₂, n is 0 to 100, a is 0 to 2, R, R¹, and R² can be identical or different at the same silicon atom and chosen from C₁-C₁₀ alkyl; C₁-C₁₀ alkyl substituted with one or more of Cl, F, N, O or S; a phenyl; C₇-C₁₆ alkylaryl; C₇-C₁₆ arylalkyl; C₂-C₂₀-polyalkylene ether; or a combination of two or more thereof. In yet another aspect, R² is chosen from OH, C₁-C₈ alkoxy, C₂-C₁₈ alkoxyalkyl, alkoxyaryl, oximoalkyl, oximoaryl, enoxyalkyl, enoxyaryl, aminoalkyl, aminoaryl, carboxyalkyl, carboxyaryl, amidoalkyl, amidoaryl, carbamatoalkyl, carbamatoaryl, or a combination of two or more thereof, and Z is a bond, a divalent unit selected from the group of a C₁-C₁₄ alkylene, or O.

In one embodiment, the present invention provides a curable composition according to any previous embodiment, wherein the crosslinker component (B) is chosen from tetraethylorthosilicate (TEOS); methyltrimethoxysilane (MTMS); vinyltrimethoxysilane; methylvinyldimethoxysilane; dimethyldimethoxysilane; dimethyldiethoxysilane; vinyltriethoxysilane; tetra(n-propyl)orthosilicate; tris(methylethylketoximo)vinylsilane; tris(methylethylketoximo)methylsilane; tris(acetamido)methylsilane; bis(acetamido)dimethylsilane; tris(N-methylacetamido)methylsilane; bis(N-methylacetamido)dimethylsilane; (N-methylacetamido)methyldialkoxysilane; tris(benzamido)methylsilane; tris(propenoxy)methylsilane; alkyldialkoxyamidosilanes; alkylalkoxybisamidosilanes; methylethoxybis(N-methylbenzamido)silane; methylethoxydibenzamidosilane; methyldimethoxy(ethylmethylketoximo)silane; bis(ethylmethylketoximo)methylmethoxysilane; (acetaldoximo)methyldimethoxysilane; (N-methylcarbamato)methyldimethoxysilane; (N-methylcarbamato) ethyldimethoxy silane; (isopropenoxy)methyldimethoxysilane; (isopropenoxy)trimethoxysilane; tris(isopropenoxy)methylsilane; (but-2-en-2-oxy)methyldimethoxysilane; (1-phenylethenoxy)methyldimethoxysilane; 2-((1-carboethoxy)propenoxy) methyldimethoxysilane; bis(N-methylamino)methylmethoxysilane; (N-methylamino)vinyldimethoxysilane; tetrakis(N,N-diethylamino)silane; methyldimethoxy(N-methylamino)silane; methyltris(cyclohexylamino)silane; methyldimethoxy(N-ethylamino)silane; dimethylbis(N,N-dimethylamino)silane; methyldimethoxy(N-isopropylamino)silane dimethylbis(N,N-diethylamino)silane; ethyldimethoxy(N- ethylpropionamido)silane; methyldimethoxy(N-methylacetamido)silane; methyltris(N-methylacetamido)silane; ethyldimethoxy(N-methylacetamido)silane; methyltris(N-methylbenzamido)silane; methylmethoxybis(N- methylacetamido)silane; methyldimethoxy(ε-caprolactamo)silane; trimethoxy(N-methylacetamido)silane; methyldimethoxy(O-ethylacetimidato)silane; methyldimethoxy(O-propylacetimidato)silane; methyldimethoxy(N,N′,N′- trimethylureido)silane; methyldimethoxy(N-allyl-N′,N′-dimethylureido)silane; methyldimethoxy(N-phenyl-N′,N′-dimethylureido)silane; methyldimethoxy(isocyanato)silane; dimethoxydiisocyanatosilane; methyldimethoxyisothiocyanatosilane; methylmethoxydiisothiocyanatosilane; methyltriacetoxysilane; methylmethoxydiacetoxysilane; methylethoxydiacetoxysilane; methylisopropoxydiacetoxysilane; methyl(n-propoxy)diacetoxysilane; methyldimethoxyacetoxysilane; methyldiethoxyacetoxysilane; methyldiisopropoxyacetoxysilane; methyldi(n-propoxy)acetoxysilane; or the condensates thereof; or a combination of two or more thereof.

In one embodiment, the curable composition is free of any adhesion promoters. In another embodiment, the curable composition comprises an adhesion promoter.

In one embodiment, the present invention provides a curable composition according to any previous embodiment, wherein the adhesion promoter component (D) is chosen from an (aminoalkyl)trialkoxysilane, an (aminoalkyl)alkyldialkoxysilane, a bis(trialkoxysilylalkyl)amine, a tris(trialkoxysilylalkyl)amine, a tris(trialkoxysilylalkyl)cyanuarate, a tris(trialkoxysilylalkyl)isocyanurate, an (epoxyalkyl)trialkoxysilane, an (epoxyalkylether)trialkoxysilane, or a combination of two or more thereof.

In one embodiment, the present invention provides a curable composition according to any previous embodiment, wherein the composition comprises about 1 to about 10 wt. % of the crosslinker component (B) based on 100 wt. % of the polymer component (A).

In one embodiment, the present invention provides a curable composition according to any previous embodiment, wherein the crosslinker component (B) is chosen from a silane or a siloxane, the silane or siloxane having two or more reactive groups that can undergo hydrolysis and/or condensation reaction with polymer (A) or on its own in the presence of water and component (F).

In one embodiment, the present invention provides a curable composition according to any previous embodiment, wherein the polymer component (A) is chosen from a polyorganosiloxane comprising divalent units of the formula [R₂SiO] in the backbone, wherein R is chosen from C₁-C₁₀ alkyl; C₁-C₁₀ alkyl substituted with one or more of Cl, F, N, O or S; phenyl; C₇-C₁₆ alkylaryl; C₇-C₁₆ arylalkyl; C₂-C₂₀ polyalkylene ether; or a combination of two or more thereof.

In one embodiment, the present invention provides a curable composition according to any previous embodiment, wherein the catalyst (C) is present in an amount of from about 0.1 to about 7 wt. pt. per 100 wt. pt. of component (A).

In one embodiment, the present invention provides a curable composition according to any previous embodiment, wherein the composition is provided as a one-part composition.

In one embodiment, the present invention provides a curable composition according to any previous embodiment, wherein the composition comprises 100 wt. % of component (A), 0.1 to about 10 wt. % of at least one crosslinker (B), 0.01 to about 7 wt. % of a catalyst (C), 0 to about 5 wt. % of an adhesion promoter (D), 0 to about 70 wt. pt. of component (E), 0.01 to about 8 wt. % of component (F) whereby this composition can be stored in the absence of humidity and is curable in the presence of humidity upon exposure to ambient air.

In one embodiment, the present invention provides a curable composition according to any previous embodiment, wherein the composition is a two-part composition comprising: (i) a first portion comprising the polymer component (A), optionally the filler component (E), and optionally the accelerator (F); and (ii) a second portion comprising the crosslinker (B), the catalyst (C), optionally, the adhesion promoter (D), optionally auxiliary component comprising an organo-functional silicon compound and/or low molecular weight organic polymer or high boiling solvents (G), whereby (i) and (ii) are stored separately until applied for curing by mixing of the components (i) and (ii).

According to one embodiment, portion (i) comprises 100 wt. % of component (A), and 0 to 70 wt. pt. of component (E); and portion (ii) comprises 0.1 to 10 wt. pt. of at least one crosslinker (B), 0.01 to 7 wt. pt. of a catalyst (C), 0 to 5 pt. wt. of an adhesion promoter (D), and 0.02 to 3 pt. wt. component (F).

In one embodiment, the present invention provides a curable composition according to any previous embodiment, wherein the composition is a two-part composition comprising: (i) a first portion comprising the polymer (A), the crosslinker (B), optionally the filler component (E), and optionally the acidic compound (F); and (ii) a second portion comprising the catalyst (C), optionally an organo-functional silicon compound and/or low molecular weight organic polymer or high boiling solvents (G), whereby (i) and (ii) are stored separately until applied for curing by mixing of the components (i) and (ii).

In another aspect, the present invention provides, a composition for forming a cured polymer composition comprising (A) a polymer having at least a reactive silyl group, where the polymer is free of siloxane bonds; (B) a crosslinker or chain extender chosen from an alkoxysilane, an alkoxysiloxane, an oximosilane, an oximosiloxane, an enoxysilane, an enoxysiloxane, an aminosilane, an aminosiloxane, a carboxysilane, a carboxysiloxane, an alkylamidosilane, an alkylamidosiloxane, an arylamidosilane, an arylamidosiloxane, an alkoxyaminosilane, an alklarylaminosiloxane, an alkoxycarbamatosilane, an alkoxycarbamatosiloxane, the condensates thereof, and combinations of two or more thereof; and (C) a catalyst comprising an amino ester.

The cure chemistry of these moisture-curable compositions can vary based upon the nature of the polymers and their moisture-curable groups. For example, alkoxysilyl groups first hydrolyze to give silanol functionalities, which then condense with the extrusion of water to give the siloxane network. Such compositions typically comprise an alkoxysilyl- or silanol-functional polymer and a crosslinking agent. Tri- and tetraalkoxysilanes are commonly used as crosslinking agents and will react with water or directly with silanol groups to crosslink the system.

### DETAILED DESCRIPTION

The present invention provides a curable composition employing an amino ester as a condensation catalyst. Compositions comprising such amino ester catalysts exhibit good curing properties and can even exhibit similar or superior curing properties compared to compositions employing organotin compounds, such as DBTDL, in terms of accelerating moisture-assisted condensation curing of silicones to result in cross-linked silicones that can be used as sealants and RTVs (Room-Temperature Vulcanized Rubber). Further, the compositions comprising such amino ester catalysts also exhibit improved storage stability.

As used herein, “alkyl” includes straight, branched, and cyclic alkyl groups. Specific and non-limiting examples of alkyls include, but are not limited to, methyl, ethyl, propyl, isobutyl, ethyl-hexyl, etc.

As used herein, “substituted alkyl” includes an alkyl group that contains one or more substituent groups that are inert under the process conditions to which the compound containing these groups is subjected. The substituent groups also do not substantially interfere with the process. As used herein, unsubstituted means the particular moiety carries hydrogen atoms on its constituent atoms, e.g. CH₃ for unsubstituted methyl. Substituted means that the group can carry typical functional groups known in organic chemistry.

As used herein, “aryl” includes a non-limiting group of any aromatic hydrocarbon from which one hydrogen atom has been removed. An aryl may have one or more aromatic rings, which may be fused, connected by single bonds or other groups. Specific and non-limiting examples of aryls include, but are not limited to, tolyl, xylyl, phenyl, naphthalenyl, etc.

As used herein, “substituted aryl” includes an aromatic group substituted as set forth in the above definition of “substituted alkyl.” Similar to an aryl, a substituted aryl may have one or more aromatic rings, which may be fused, connected by single bonds or other groups; however, when the substituted aryl has a heteroaromatic ring, the free valence in the substituted aryl group can be a heteroatom (such as nitrogen) of the heteroaromatic ring instead of a carbon. In one embodiment, substituted aryl groups herein contain 1 to about 30 carbon atoms.

As used herein “aralkyl” include an alkyl group substituted by aryl groups.

As used herein, “alkenyl” includes any straight, branched, or cyclic alkenyl group containing one or more carbon-carbon double bonds, where the point of substitution can be either a carbon-carbon double bond or elsewhere in the group. Specific and non-limiting examples of alkenyls include, but are not limited to, vinyl, propenyl, allyl, methallyl, ethylidenyl norbornane, etc.

As used herein, “alkynyl” includes any straight, branched, or cyclic alkynyl group containing one or more carbon-carbon triple bonds, where the point of substitution can be either at a carbon-carbon triple bond or elsewhere in the group.

As used herein, “unsaturated” refers to one or more double or triple bonds. In one embodiment, it refers to carbon-carbon double or triple bonds.

As used herein, the terms “alkylene”, “cycloalkylene”, “alkynylene”, “alkenylene”, and “arylene” alone or as part of another substituent refers to a divalent radical derived from an alkyl, cycloalkyl, heteroalkyl, alkynyl, alkenyl, or aryl group, respectively. The respective radicals can be substituted or unsubstituted, linear or branched.

As used herein, “silicon-containing alkyl,” “silicon-containing aryl,” etc., include compounds comprising the group —SiR₃, where R may be the same or different and is chosen from the group containing an alkyl, a cycloalkyl, a heteroalkyl, a heterocycloalkyl, an aryl, a heteroaryl, an alkoxy, or a hydroxy.

As used herein, “heteroalkyl,” “heteroaryl,” etc. include compounds comprising a hetero atom such as O, N, P, S, etc.

In one embodiment, the present invention provides a curable composition comprising a polymer component (A) comprising a reactive terminal silyl group; a crosslinker component (B); a catalyst component (C) comprising an amino ester; optionally an adhesion promoter component (D); an optional filler component (E); optionally an acidic compound (F), and optionally auxiliary component comprising an organo-functional silicon compound and/or low molecular weight organic polymer or high boiling solvents (G).

In another embodiment, the present invention provides a curable composition comprising a polymer component (A) comprising a hydridosilyl group; a catalyst component (C) comprising an amino ester; and optionally auxiliary components (G).

The polymer component (A) may be a liquid- or solid-based polymer having a reactive terminal silyl group. The polymer component (A) is not particularly limited and may be chosen from any cross-linkable polymer as may be desired for a particular purpose or intended use. Non-limiting examples of suitable polymers for the polymer component (A) include polyorganosiloxanes (A1) or organic polymers free of siloxane bonds (A2), wherein the polymers (A1) and (A2) comprise reactive terminal silyl groups. In one embodiment, the polymer component (A) may be present in an amount of from about 10 to about 90 wt. % of the curable composition. In one embodiment, the curable composition comprises about 100 pt. wt. of the polymer component (A).

As described above, the polymer component (A) may include a wide range of polyorganosiloxanes. In one embodiment, the polymer component may comprise one or more polysiloxanes and copolymers of formula (1):

[R¹_{c}R²_{3-c}Si-Z-]ₙ-X-Z-SiR¹_{c}R²_{3-c} (1)

R¹ may be chosen from linear or branched alkyl, linear or branched heteroalkyl, cycloalkyl, heterocycloalkyl, aryl, heteroaryl, linear or branched aralkyl, linear or branched heteroaralkyl, or a combination of two or more thereof. In one embodiment, R¹ may be chosen from C₁-C₁₀ alkyl; C₁-C₁₀ alkyl substituted with one or more of Cl, F, N, O or S; phenyl; C₇-C₁₆ alkylaryl; C₇-C₁₆ arylalkyl; C₂-C₂₀ polyalkylene ether; or a combination of two or more thereof. Exemplary groups are methyl, trifluoropropyl, and/or phenyl groups.

R²may be a group reactive to protic agents such as water. Exemplary groups for R² include OH, alkoxy, alkenyloxy, alkyloximo, alkylcarboxy, arylcarboxy, alkylamido, arylamido, or a combination of two or more thereof. In one embodiment, R² is chosen from OH, C₁-C₈ alkoxy, C₂-C₁₈ alkoxyalkyl, amino, alkenyloxy, alkyloximo, alkylamino, arylamino, alkylcarboxy, arylcarboxy, alkylamido, arylamido, alkylcarbamato, arylcarbamato, or a combination of two or more thereof.

Z may be a bond, a divalent linking unit selected from the group of O, hydrocarbons which can contain one or more O, S, or N atom, guanidine-containing, urethane, ether, ester, urea units or a combination of two or more thereof. If the linking group Z is a hydrocarbon group, then Z is linked to the silicon atom over a silicon-carbon bond. In one embodiment, Z is chosen from a C₁-C₁₄ alkylene.

X is chosen from a polyurethane; a polyester; a polyether; a polycarbonate; a polyolefin; a polyesterether; and a polyorganosiloxane having units of R¹₃SiO_{1/2}, R¹₂SiO, R¹SiO_{3/2}, and/or SiO₂, where R¹ is defined as above. X may be a divalent or multivalent polymer unit selected from the group of siloxy units linked over oxygen or hydrocarbon groups to the terminal silyl group comprising the reactive group R² as described above, polyether, alkylene, isoalkylene, polyester, or polyurethane units linked over hydrocarbon groups to the silicon atom comprising one or more reactive groups R² as described above. The hydrocarbon group X can contain one or more heteroatoms such as N, S, O, or P forming guanidine-containing esters, ethers, urethanes, esters, and/or ureas. In one embodiment, the average polymerization degree (Pₐ) of X should be more than 6, e.g. polyorganosiloxane units of R¹₃SiO_{1/2}, R¹₂SiO, R¹SiO_{3/2}, and/or SiO₂. In formula (2), n is 0 to 100; desirably 1, and c is 0 to 2, desirably 0 to 1.

Non-limiting examples of the components for unit X include polyoxyalkylene polymers such as polyoxyethylene, polyoxypropylene, polyoxybutylene, polyoxyethylene-polyoxypropylene copolymer, polyoxytetramethylene, or polyoxypropylene-polyoxybutylene copolymer; ethylene-propylene copolymer, polyisobutylene, polychloroprene, polyisoprene, polybutadiene, copolymer of isobutylene and isoprene, copolymers of isoprene or butadiene and acrylonitrile and/or styrene, or hydrocarbon polymers such as hydrogenated polyolefin polymers produced by hydrogenating these polyolefin polymers; polyester polymer manufactured by a condensation of dibasic acid such as adipic acid or phthalic acid and glycol, or ring-opening polymerization of lactones; polyacrylic acid ester produced by radical polymerization of a monomer such as C₂-C₈-alkyl acrylates, vinyl polymers, e.g., acrylic acid ester copolymer of acrylic acid ester such as ethyl acrylate or butyl acrylate and vinyl acetate, acrylonitrile, methyl methacrylate, acrylguanidine-containing, or styrene; graft polymer produced by polymerizing the above organic polymer with a vinyl monomer; polycarbonates; polysulfide polymer; polyguanidine-containing polymer such as Nylon 6 produced by ring-opening polymerization of E-caprolactam, Nylon 6-6 produced by polycondensation of hexamethylenediamine and adipic acid, etc., Nylon 12 produced by ring-opening polymerization of E-laurolactam, copolymeric polyguanidine-containings polyurethanes or polyureas.

Particularly suitable polymers include, but are not limited to, polysiloxanes, polyoxyalkylenes, saturated hydrocarbon polymers such as polyisobutylene, hydrogenated polybutadiene and hydrogenated polyisoprene, or polyethylene, polypropylene, polyesters, polycarbonates, polyurethanes, polyurea polymers and the like. Furthermore, saturated hydrocarbon polymer, polyoxyalkylene polymer, and vinyl copolymer are particularly suitable due to their low glass transition temperature which provide a high flexibility at low temperatures, i.e., below 0° C.

The reactive silyl groups in formula (1) can be introduced by employing silanes containing a functional group which has the ability to react by known methods with unsaturated hydrocarbons via hydrosilylation, or reaction of SiOH, aminoalkyl or -aryl, HOOC-alkyl or -aryl, HO-alkyl or -aryl, HS-alkyl or -aryl, Cl(O)C-alkyl or -aryl, epoxyalkyl or epoxycycloalkyl groups in the prepolymer to be linked to a reactive silyl group via condensation or ring-opening reactions. Examples of the main embodiments include the following: (i) siloxane prepolymers having a SiOH group that can undergo a condensation reaction with a silane (LG)SiR¹_{c}R²_{3-c} whereby a siloxy bond ≡Si—O—SiR¹_{c}R²_{3-c} is formed while the addition product of the leaving group (LG) and hydrogen is released (LG-H); (ii) silanes having an unsaturated group that is capable of reacting via hydrosilylation or radical reaction with a SiH group or radically activated groups of a silane such as SiH or an unsaturated group; and (iii) silanes including organic or inorganic prepolymers having OH, SH, amino, epoxy, —COCl, —COOH groups, which can react complementarily with epoxy, isocyanato, OH, SH, cyanato, carboxylic halogenides, reactive alkylhalogenides, lactones, lactams, or amines, that is to link the reactive prepolymer with the organofunctional silanes to yield a silyl functional polymer.

Silanes suitable for method (i) include alkoxysilanes, especially tetraalkoxysilanes, di- and trialkoxysilanes, di- and triacetoxysilanes, di- and triketoximosilanes, di- and trialkenyloxysilanes, di- and tricarbonamidosilanes, wherein the remaining residues at the silicon atom of the silane are substituted or unsubstituted hydrocarbons. Other non-limiting silanes for method (i) include alkyltrialkoxysilanes, such as vinyltrimethoxysilane, methyltrimethoxysilane, propyltrimethoxysilane, aminoalkyltrimethoxysilane, ethyltriacetoxysilane, methyl- or propyltriacetoxysilane, methyltributanonoximosilane, methyltripropenyloxysilane, methyltribenzamidosilane, or methyltriacetamidosilane. Prepolymers suitable for reaction under method (i) are SiOH-terminated polyalkylsiloxanes, which can undergo a condensation reaction with a silane having hydrolyzable groups attached to the silicon atom. Exemplary SiOH-terminated polyalkyldisiloxanes include polydimethylsiloxanes.

Suitable silanes for method (ii) include alkoxysilanes, especially trialkoxysilanes (HSi(OR)₃) such as trimethoxysilane, triethoxysilane, methyldiethoxysilane, methyldimethoxysilane, and phenyldimethoxysilane. Hydrogenchlorosilanes are in principle possible but are less desirable due to the additional replacement of the halogen through an alkoxy, acetoxy group, etc. Other suitable silanes include organofunctional silanes having unsaturated groups which can be activated by radicals, such as vinyl, allyl, mercaptoalkyl, or acrylic groups. Non-limiting examples include vinyltrimethoxysilane, mercaptopropyltrimethoxysilane, and methacryloxypropyltrimethoxysilane. Prepolymers suitable for reaction under method (ii) include vinyl-terminated polyalkylsiloxanes, preferably polydimethylsiloxanes, hydrocarbons with unsaturated groups which can undergo hydrosilylation or can undergo radically induced grafting reactions with a corresponding organofunctional group of a silane comprising, for example, unsaturated hydrocarbon or a SiH group.

Another method for introducing silyl groups into hydrocarbon polymers can be the copolymerization of unsaturated hydrocarbon monomers with the unsaturated groups of silanes. The introduction of unsaturated groups into a hydrocarbon prepolymer may include, for example, the use of alkenyl halogenides as chain stopper after polymerization of the silicon free hydrocarbon moiety.

Desirable reaction products between the silanes and prepolymers include the following structures: —SiR¹₂O—SiR¹₂-CH₂—CH₂—SiR¹_{c}R²_{3-c}, or (hydrocarbon)-[Z-SiR¹_{c}R²_{3-c}]ₙ. Suitable silanes for method (iii) include, but are not limited to, alkoxysilanes, especially silanes having organofunctional groups to be reactive to —OH, —SH, amino, epoxy, —COCl, or —COOH.

In one embodiment, these silanes have an isocyanatoalkyl group such as gamma-isocyanatopropyltrimethoxysilane, gamma-isocyanatopropylmethyldimethoxysilane, gamma-isocyanatopropyltriethoxysilane, gamma-glycidoxypropylethyldimethoxysilane, gamma-glycidoxypropyltrimethoxysilane, gamma-glycidoxypropyltriethoxysilane, beta-(3,4-epoxycyclohexyl)ethyltrimethoxysilane, beta-(3,4-epoxycyclohexyl)ethyltriethoxysilane, epoxylimonyltrimethoxysilane, N-(2-aminoethyl)-aminopropyltrimethoxysilane, gamma-aminopropyltriethoxysilane, gamma-aminopropyltrimethoxysilane, gamma-aminopropylmethyldimethoxysilane, gamma-aminopropylmethyldiethoxysilane, etc.

In one embodiment, it is desirable to select either blocked amines or isocyanates (Z′-X)ₙ-Z′ for carrying out first a complete mixing and then the following coupling reaction. Examples of blocking agents are disclosed in EP 0947531 and other blocking procedures that employ heterocyclic nitrogen compounds such as caprolactam or butanone oxime, or cyclic ketones referred to in U.S. Pat. No. 6,827,875 both of which are incorporated herein by reference in their entirety.

Examples of suitable prepolymers for a reaction under method (iii) include, but are not limited to, polyalkylene oxides having OH groups, in one embodiment with a high molecular weight (M_{w}, weight-average molecular weight>6000 g/mol) and a polydispersity M_{w}/Mₙ of less than 1.6; urethanes having remaining NCO groups, such as NCO functionalized polyalkylene oxides, especially blocked isocyanates. Prepolymers selected from the group of hydrocarbons having —OH, —COOH, amino, epoxy groups, which can react complementarily with an epoxy, isocyanato, amino, carboxyhalogenide or halogenalkyl group of the corresponding silane having further reactive groups useful for the final cure.

Suitable isocyanates for the introduction of a NCO group into a polyether may include toluene diisocyanate, diphenylmethane diisocyanate, or xylene diisocyanate, or aliphatic polyisocyanate such as isophorone diisocyanate, or hexamethylene diisocyanate.

The polymerization degree of the unit X depends on the requirements of viscosity and mechanical properties of the cured product. If X is a polydimethylsiloxane unit, the average polymerization degree based on the number average molecular weight Mₙ is preferably 7 to 5000 siloxy units, preferably 200 to 2000 units. In order to achieve a sufficient tensile strength of >5 MPa, an average polymerization degree Pₐ of >250 is suitable whereby the polydimethylsiloxanes have a viscosity of more than 300 mPa·s at 25° C. If X is a hydrocarbon unit other than a polysiloxane unit, the viscosity with respect to the polymerization degree is much higher.

Examples of the method for synthesizing a polyoxyalkylene polymer include, but are not limited to, a polymerization method using an alkali catalyst such as KOH, a polymerization method using a metal-porphyrin complex catalyst such as a complex obtained by reacting an organoaluminum compound, a polymerization method using a composite metal cyanide complex catalyst disclosed, e.g., in U.S. Pat. Nos. 3,427,256; 3,427,334; 3,278,457; 3,278,458; 3,278,459; 3,427,335; 6,696,383; and 6,919,293.

If the group X is selected from hydrocarbon polymers, then polymers or copolymers having isobutylene units are particularly desirable due to its physical properties such as excellent weatherability, excellent heat resistance, and low gas and moisture permeability.

Examples of the monomers include olefins having 4 to 12 carbon atoms, vinyl ether, aromatic vinyl compound, vinylsilanes, and allylsilanes. Examples of the copolymer component include 1-butene, 2-butene, 2-methyl-1-butene, 3-methyl-1-butene, pentene, 4-methyl-1-pentene, hexene, vinylcyclohexene, methyl vinyl ether, ethyl vinyl ether, isobutyl vinyl ether, styrene, alpha-methylstyrene, dimethylstyrene, beta-pinene, indene, and for example, but not limited to, vinyltrialkoxysilanes, e.g. vinyltrimethoxysilane, vinylmethyldichlorosilane, vinyldimethylmethoxysilane, divinyldichlorosilane, divinyldimethoxysilane, allyltrichlorosilane, allylmethyldichlorosilane, allyldimethylmethoxysilane, diallyldichlorosilane, diallyldimethoxysilane, gamma-methacryloyloxypropyltrimethoxysilane, and gamma-methacryloyloxypropylmethyldimethoxy silane.

Examples of suitable siloxane-free organic polymers include, but are not limited to, silylated polyurethane (SPUR), silylated polyester, silylated polyether, silylated polycarbonate, silylated polyolefins like polyethylene, polypropylene, silylated polyesterether and combinations of two or more thereof The siloxane-free organic polymer may be present in an amount of from about 10 to about 90 wt. % of the composition or about 100 pt. wt.

In one embodiment, the polymer component (A) may be silylated polyurethane (SPUR). Such moisture curable compounds are known in the art in general and can be obtained by various methods including (i) reacting an isocyanate-terminated polyurethane (PUR) prepolymer with a suitable silane, e.g., one possessing both hydrolyzable functionality at the silicon atom, such as, alkoxy, etc., and secondly active hydrogen-containing functionality such as mercaptan, primary or secondary amine, preferably the latter, etc., or by (ii) reacting a hydroxyl-terminated PUR (polyurethane) prepolymer with a suitable isocyanate-terminated silane, e.g., one possessing one to three alkoxy groups. The details of these reactions, and those for preparing the isocyanate-terminated and hydroxyl-terminated PUR prepolymers employed therein can be found in, amongst others: U.S. Pat. Nos. 4,985,491; 5,919,888; 6,207,794; 6,303,731; 6,359,101; and 6,515,164, and published U.S. Patent Publication Nos. 2004/0122253 and US 2005/0020706 (isocyanate-terminated PUR prepolymers); U.S. Pat. Nos. 3,786,081 and 4,481,367 (hydroxyl-terminated PUR prepolymers); U.S. Pat. Nos. 3,627,722; 3,632,557; 3,971,751; 5,623,044; 5,852,137; 6,197,912; and 6,310,170 (moisture-curable SPUR (silane modified/terminated polyurethane) obtained from reaction of isocyanate-terminated PUR prepolymer and reactive silane, e.g., aminoalkoxysilane); and, U.S. Pat. Nos. 4,345,053; 4,625,012; 6,833,423; and published U.S. Patent Publication 2002/0198352 (moisture-curable SPUR obtained from reaction of hydroxyl-terminated PUR prepolymer and isocyanatosilane). The entire contents of the foregoing U.S. patent documents are incorporated by reference herein. Other examples of moisture-curable SPUR materials include those described in U.S. Pat. No. 7,569,653, the disclosure of which is incorporated by reference in its entirety.

In one embodiment, the polymer component (A) may be a polymer of formula (2):

R²_{3-c}R¹_{c}Si-Z-[R₂SiO]ₓ[R¹₂SiO]_{y}-Z-SiR¹_{c}R²_{3-c} (2)

where R¹, R², Z, and c are defined as above with respect to formula (2); R is C₁-C₆ alkyl (an exemplary alkyl being methyl); x is 0 to about 10,000, in one embodiment from 11 to about 2500; and y is 0 to about 10,000; preferably 0 to 500. In one embodiment, Z in a compound of formula (2) is a bond or a divalent C₁-C₁₄ alkylene group, especially preferred is —C₂H₄—.

In one embodiment, the polymer component (A) may be a polyorganosiloxane of the formula (3):

R²_{3-c-d}SiR³_{c}R⁴_{d}-[OSiR³R⁴]ₓ-[OSiR³R⁴]_{y}-OSiR³_{c}R⁴_{f}R²_{3-3-f} (3)

R³ and R⁴ can be identical or different on the same silicon atom and are chosen from hydrogen; C₁-C₁₀ alkyl; C₁-C₁₀ heteroalkyl, C₃-C₁₂ cycloalkyl; C₂-C₃₀ heterocycloalkyl; C₆-C₁₃ aryl; C₇-C₃₀ alkylaryl; C₇-C₃₀ arylalkyl; C₄-C₁₂ heteroaryl; C₅-C₃₀ heteroarylalkyl; C₅-C₃₀ heteroalkylaryl; C₂-C₁₀₀ polyalkylene ether; or a combination of two or more thereof. R², c, x, and y are as defined above; d is 0, 1, or 2; e is 0, 1, or 2; and f is 0, 1, or 2.

Non-limiting examples of suitable polysiloxane-containing polymers (A1) include, for example, silanol-stopped polydimethylsiloxane, silanol or alkoxy-stopped polyorganosiloxanes, e.g., methoxystopped polydimethylsiloxane, alkoxy-stopped polydimethylsiloxane-polydiphenylsiloxane copolymer, and silanol or alkoxy-stopped fluoroalkyl-substituted siloxanes such as poly(methyl 3,3,3-trifluoropropyl)siloxane and poly(methyl 3,3,3-trifluoropropyl)siloxane-polydimethyl siloxane copolymer. The polyorganosiloxane component (A1) may be present in an amount of about 10 to about 90 wt. % of the composition or 100 pt. wt. In one preferred embodiment, the polyorganosiloxane component has an average chain length in the range of about 10 to about 2500 siloxy units, and the viscosity is in the range of about 10 to about 500,000 mPa·s at 25° C.

Alternatively, the composition may include silyl-terminated organic polymers (A2) that are free of siloxane units, and which undergo curing by a condensation reaction comparable to that of siloxane containing polymers (A1). Similar to the polyorganosiloxane polymer (A1), the organic polymers (A2) that are suitable as the polymer component (A) include a terminal silyl group. In one embodiment, the terminal silyl group may be of the formula (4):

—SiR¹_{d}R²_{3-d} (4)

where R¹, R², and d are as defined above.

The polysiloxane composition may further include a crosslinker or a chain extender as component (B). In one embodiment, the crosslinker is of the formula (5):

R¹_{d}SiR²_{4-d} (5)

wherein R¹, R², and d are as defined above. Alternatively, the crosslinker component may be a condensation product of formula (5) wherein one or more but not all R² groups are hydrolyzed and released in the presence of water and then intermediate silanols undergo a condensation reaction to give a Si—O—Si bond and water. The average polymerization degree can result in a compound having 2 to 10 Si units.

In one embodiment, the crosslinker is an alkoxysilane having a formula (6):

R³_{d}(R¹O)_{4-d}Si, (6)

wherein R¹, R³, and d are defined as above.

In another embodiment, the crosslinker is an acetoxysilane having a formula (7):

(R³_{d}(R¹CO₂)_{4-d}Si, (7)

wherein R¹, R³, and d are defined as above.

In still another embodiment, the crosslinker is an oximosilane having a formula (8)

R³_{d}(R¹R⁴C═N—O)_{4-d}Si, (8)

where R¹, R³, R⁴, and d are defined as above.

As used herein, the term crosslinker includes a compound including an additional reactive component having at least two hydrolysable groups and less than three silicon atoms per molecule not defined under (A). In one embodiment, the crosslinker or chain extender may be chosen from an alkoxysilane, an alkoxysiloxane, an oximosilane, an oximosiloxane, an enoxysilane, an enoxysiloxane, an aminosilane, an aminosiloxane, a carboxysilane, a carboxysiloxane, an alkylamidosilane, an alkylamidosiloxane, an arylamidosilane, an arylamidosiloxane, an alkoxyaminosilane, an alkylarylaminosiloxane, an alkoxycarbamatosilane, an alkoxycarbamatosiloxane, an imidatosilane, a ureidosilane, an isocyanatosilane, a isothiocyanatosilane, the condensates thereof, a hydridosilane, a hydridosiloxane (organosiloxane monomer, oligomer and/or polymer having, per molecule, at least one reactive ≡SiH unit), and combinations of two or more thereof. Examples of suitable cross-linkers include, but are not limited to, tetraethylorthosilicate (TEOS); methyltrimethoxysilane (MTMS); methyltriethoxysilane; vinyltrimethoxysilane; vinyltriethoxysilane; methylphenyldimethoxysilane; 3,3,3-trifluoropropyltrimethoxysilane; methyltriacetoxysilane; vinyltriacetoxysilane; ethyltriacetoxysilane; di-butoxydiacetoxysilane; phenyltripropionoxysilane; methyltris(methylethylketoximo)silane; vinyltris(methylethylketoximo)silane; 3,3,3-trifluoropropyltris(methylethylketoximo)sane; methyltris(isopropenoxy)silane; vinyltris(isopropenoxy)silane; ethylpolysilicate; dimethyltetraacetoxydisiloxane; tetra-n-propylorthosilicate; methyldimethoxy(ethylmethylketoximo)silane; methylmethoxybis(ethylmethylketoximo)silane; methyldimethoxy(acetaldoximo)silane; methyldimethoxy(N-methylcarbamato)silane; ethyldimethoxy(N-methylcarbamato)silane; methyldimethoxyisopropenoxysilane; trimethoxyisopropenoxysilane; methyltriisopropenoxysilane; methyldimethoxy(but-2-en-2-oxy)silane; methyldimethoxy(l-phenylethenoxy)silane; methyldimethoxy-2-(1-carboethoxypropenoxy)silane; methylmethoxydi(N-methylamino)silane; vinyldimethoxy(methylamino)silane; tetra-N,N-diethylaminosane; methyldimethoxy(methylamino)silane; methyltri(cyclohexylamino)silane; methyldimethoxy(ethylamino)silane; dimethyldi(N,N-dimethylamino)silane; methyldimethoxy(isopropylamino)silane; dimethyldi(N,N-diethylamino)silane; ethyldimethoxy(N-ethylpropionamido)silane; methyldimethoxy(N-methylacetamido)silane; methyltris(N-methylacetamido)silane; ethyldimethoxy(N-methylacetamido)silane; methyltris(N-methy lbenzamido)silane; methylmethoxybis(N-methylacetamido)silane; methyldimethoxy(caprolactamo)silane; trimethoxy(N-methylacetamido)silane; methyldimethoxy(ethylacetimidato)silane; methyldimethoxy(propylacetimidato)silane; methyldimethoxy(N,N′,N′-trimethylureido)silane; methyldimethoxy(N-allyl-N′,N′-dimethylureido)silane; methyldimethoxy(N-phenyl-N′,N′-dimethylureido)silane; methyldimethoxyisocyanatosilane; dimethoxydiisocyanatosilane; methyldimethoxyisothiocyanatosilane; methylmethoxydiisothiocyanatosilane, the condensates thereof, or combinations of two or more thereof. In one embodiment, the crosslinker may be present in an amount from about 1 to about 10 wt. % of the composition or from about 0.1 to about 10 pt. wt. per 100 pt. wt. of the polymer component (A). In another embodiment, the crosslinker may be present in an amount from about 0.1 to about 5 pt. wt. per 100 pt. wt. of the polymer component (A). In still another embodiment, the crosslinker may be present in an amount from about 0.5 to about 3 pt. wt. per 100 pt. wt. of the polymer component (A). Here as elsewhere in the specification and claims, numerical values may be combined to form new or undisclosed ranges.

The composition can include a chain extender. The chain extenders can be reactive or non-reactive and can be chosen from a variety of compounds including, but not limited to, organo-functional silicon compounds, (e.g., hydroxyl, carboxylic acid, ester, polyether, amide, amine, alkyl, and/or aromatic grafted/capped siloxane), an alkyl stopped siloxone such as, for example, methyl stopped PDMS, nonreactive organic polymers, or a combination of two or more thereof The organo-functional silicon compounds can be referred to as organosilicon compounds. The organosilicon compounds can be linear or branched. Examples of suitable organo-functional silicon compounds include, but are not limited to hydride terminated, vinyl terminated, hydroxyl terminated, and/or amino terminated siloxane. In one embodiment, the extender is a organo-functional polydimethylsiloxane such as, for example, hydride terminated polydimethylsiloxane, silanol terminated polydimethylsiloxane, vinyl terminated polydimethylsiloxane, and/or amino terminated polydimethyl siloxane.

In one embodiment, the chain extender is an organosilicon compound having hydrolyzable groups. Examples of suitable hydrolyzable groups include, but are not limited to an alkoxy group, an alkoxyalkoxy group, or a combination of two or more thereof. Non-limiting examples of suitable hydrolyzable groups include methoxy, ethoxy, propoxy, isopropoxy, butoxy, methoxyethoxy, etc., and combinations of two or more thereof. Still further examples of suitable organosilicon compounds include, but are not limited to, tetraethoxysilane, tetramethoxysilane, methyltrimethoxysilane, vintlytrimethoxysilane, methyltriethoxysilane, vinyltriethoxysilane, ethylorthosilicate, propylyorthosilicate, partial hydrosylates of such compounds, etc., and combinations of two or more thereof

In one embodiment, the crosslinker is of the formula (9):

R¹_{g}SiR²_{4-q} (9),

wherein R¹ may be chosen from saturated C₁- C₁₂ alkyl (which can be substituted with one or more of a halogen (e.g., Cl, F, O, S or N atom), C₅-C₁₆ cycloalkyl, C₂-C₁₂ alkenyl, C₇-C₁₆ arylalkyl, C₇-C₁₆ alkylaryl, phenyl, C₂-C₄ polyalkylene ether, or a combination of two or more thereof. Exemplary preferred groups are methyl, trifluoropropyl and/or phenyl groups; R² may be a group reactive to protonated agents such as water and may be chosen from OH, C₁-C₈-alkoxy, C₂-C₁₈-alkoxyalkyl, amino, alkenyloxy, oximoalkyl, enoxyalkyl, aminoalkyl, carboxyalkyl, amidoalkyl, amidoaryl, carbamatoalkyl or a combination of two or more thereof. Exemplary groups for R² include OH, alkoxy, alkenyloxy, alkyloximo, alkylcarboxy, alkylamido, arylamido, or a combination of two or more thereof, and q is 0-3. Alternatively, the cross-linker component may be a condensation product of formula (6) wherein one or more but not all R² groups are hydrolyzed and released in the presence of water and then intermediate silanols undergo a condensation reaction to give a Si—O—Si bond and water. The average polymerization degree can result in a compound having 2-10 Si units.

As used herein, the term crosslinker includes a compound including an additional reactive component having at least 2 hydrolysable groups and less than 3 silicon atoms per molecule not defined under (A). In one embodiment, the crosslinker or chain extender may be chosen from an alkoxysilane, an alkoxysiloxane, an oximosilane, an oximosiloxane, an enoxysilane, an enoxysiloxane, an aminosilane, a carboxysilane, a carboxysiloxane, an alkylamidosilane, an alkylamidosiloxane, an arylamidosilane, an arylamidosiloxane, an alkoxyaminosilane, an alkaryaminosiloxane, an alkoxycarbamatosilane, an alkoxycarbamatosiloxane, an imidatosilane, a ureidosilane, an isocyanatosilane, a thioisocyanatosilane, and combinations of two or more thereof. Examples of suitable cross-linkers include, but are not limited to, tetraethylorthosilicate (TEOS); methyltrimethoxysilane (MTMS); methyltriethoxysilane; vinyltrimethoxysilane; vinyltriethoxysilane; methylphenyldimethoxysilane; 3,3,3-trifluoropropyltrimethoxysilane; methyltriacetoxysilane; vinyltriacetoxysilane; ethyltriacetoxysilane; di-butoxydiacetoxysilane; phenyltripropionoxysilane; methyltris(methylethylketoxime)silane; vinyltris(methylethylketoxime)silane; 3,3,3-trifluoropropyltris(methylethylketoxime)silane; methyltris(isopropenoxy)silane; vinyltris(isopropenoxy)silane; ethylpolysilicate; dimethyltetraacetoxydisiloxane; tetra-n-propylorthosilicate; methyldimethoxy(ethylmethylketoximo)silane; methylmethoxybis-(ethylmethylketoximo)silane; methyldimethoxy(acetaldoximo)silane; methyldimethoxy(N-methylcarbamato)silane; ethyldimethoxy(N-methylcarbamato)silane; methyldimethoxyisopropenoxysilane; trimethoxyisopropenoxysilane; methyltri-iso-propenoxysilane; methyldimethoxy(but-2-ene-2-oxy)silane; methyldimethoxy(l-phenylethenoxy)silane; methyldimethoxy-2(1-carboethoxypropenoxy)silane; methylmethoxydi-N-methylaminosilane; vinyldimethoxymethylaminosilane; tetra-N,N-diethylaminosilane; methyldimethoxymethylaminosilane; methyltricyclohexylaminosilane; methyldimethoxyethylaminosilane; dimethyldi-N,N-dimethylaminosilane; methyldimethoxyisopropylaminos ane dimethyldi-N,N-diethylaminosilane. ethyldimethoxy(N-ethylpropionamido)silane; methyldimethoxy(N-methylacetamido)silane; methyltris(N-methylacetamido)silane; ethyldimethoxy(N-methylacetamido)silane; methyltris(N-methylbenzamido)silane; methylmethoxybis(N-methylacetamido)silane; methyldimethoxy(caprolactamo)silane; trimethoxy(N-methylacetamido)silane; methyldimethoxyethylacetimidatosilane; methyldimethoxypropylacetimidatosilane; methyldimethoxy(N,N,N-trimethylureido)silane; methyldimethoxy(N-allyl-N′,N′-dimethylureido)silane; methyldimethoxy(N-phenyl-N′,N′-dimethylureido)silane; methyldimethoxyisocymatosilane; dimethoxydiisocyanatosilane; methyldimethoxythioisocymatosilane; methylmethoxydithioisocyanatosilane,or combinations of two or more thereof. The crosslinker may be present in an amount from about 1 to about 10 wt. % of the composition or from about 0.1 to about 10 pt. wt. per 100 pt. wt. of the polymer component (A).

In one embodiment, the composition can further include an organo-functional silicon compound, a low-molecular-weight organic polymer, a high-boiling-point solvent, or a combination of two or more thereof. Organo-functional silicon compounds include, but are not limited to, an organo-functional silane and/or an organo-functional siloxane. It has been found that the use of organo-functional silanes, organo-functional siloxanes, and/or low-molecular-weight organic polymers with the carboxylic acid catalyst component can enhance the properties of the composition. The compositions still exhibit good curability and adhesion as well as retaining stability under storage and not exhibiting phase separation.

The low-molecular-weight organic polymers, high-boiling-point solvents, and organo-functional silicon compounds may also be referred to herein as extenders.

Low-molecular-weight organic polymers suitable as the extender include compounds or materials having a boiling point greater than 150° C.; in one embodiment from 150° C. to 450° C. Examples of suitable low-molecular-weight compounds as the extender include, but are not limited to, polyether polyols containing repeating ether linkage -R-O-R- and have two or more hydroxyl groups as terminal functional groups, or combinations of two or more thereof. In one embodiment, polyethylene glycol can be employed as an extender.

High-boiling molecules suitable as extenders include high-boiling-point solvents having a boiling point of at least 150° C. For example, a boiling point between 150° C. and 450° C., between 225° C. and 375° C., even between 275° C. and 325° C. Examples of high-boiling-point solvents as extenders include, but are not limited to DMF, DMSO, carbitols or combinations of two or more thereof.

The organo-functional silicon compound can be chosen from a variety of compounds, including, but not limited to, carboxylic acid, ester, polyether, amide, amine, alkyl, aryl, aromatic grafted or endcapped siloxanes, organic polymers, or a combination of two or more thereof. For example, the organo-functional silicon can be an alkyl-stopped siloxane such as, for example, methyl-stopped PDMS. The organo-functional silicon compounds can be referred to as organosilicon compounds. The organosilicon compounds can be linear or branched. Examples of suitable organo-functional silicon compounds include, but are not limited to, hydrido-functional siloxanes, vinyl- functional siloxanes, hydroxyl- functional siloxanes, and amino-functional siloxanes. In one embodiment, the extender is an organo-functional polydimethylsiloxane compound such as, for example, hydride-terminated polydimethylsiloxane, silanol-terminated polydimethylsiloxane, vinyl-terminated polydimethylsiloxane, or amino-terminated polydimethylsiloxane.

In one embodiment, the composition comprises an organo-functional siloxane of the formula (10):

M Dₕ D′ₖ T_{z} T′ⱼ M (10)

where M represents R⁶₃SiO_{1/2}; D is R⁷₂SiO_{2/2}; D′ is R⁸₂SiO_{3/2}, T is R⁹SiO_{3/2}; T′ is R¹⁰SiO_{3/2}; R⁶, R⁷, R⁸, R⁹, and R¹⁰ are independently chosen from a hydrogen and a monovalent organic group, such as an alkyl group, a heteroalkyl group, an alkenyl group, a heteroalkenyl group, a cycloalkyl group, a heterocycloalkyl, an aryl group, a heteroaryl group, an aryloxy group, an aralkyl group, a heteroaralkyl group, an alkylaryl group, a heteroalkylaryl group, an epoxy group, an amino group, a mercapto group, a trifluoropropyl group, a polyalkylene oxide group, a silicon-containing alkyl group, a silicon-containing aryl group, an alkyl, aryl, alkylaryl, or aralkyl bridge formed by at least two R⁶, two R⁷, or two R⁸ groups. The values of h, k, z, and j may vary greatly depending upon the desired end viscosity of the polymers of the present invention. In one embodiment, the viscosity of the organo-functional silicon compound is between the range of about 1 centiStokes (cSt) at 25° C. to about 2,000,000 centiStokes (cSt) at 25° C. In another embodiment, the viscosity of the organo-functional silicon compound is between the range of about 1 cSt at 25° C. to about 200,000 cSt at 25° C. In yet another embodiment, the viscosity of the organo-functional silicon compound is between the range of about 1 cSt at 25° C. to about 10,000 cSt at 25° C. In yet another embodiment, the viscosity of the organo-functional silicon compound is between the range of about 1 cSt at 25° C. to about 3,000 cSt at 25° C. Here as elsewhere in the specification and claims, numerical values can be combined to form new and non-disclosed ranges. The organo-functional silicon compound comprises at least one organic group. In one embodiment, R⁶, R⁷, and R⁸ are independently chosen from a C1-C13 alkyl group, a C1-C13 alkoxygroup, a C2-C13 alkenyl group, a C2-C13 alkenyloxy group, a C3-C6 cycloalkyl group, a C3-C6 cycloalkoxy group, a C6-C14 aryl group, a C6-C10 aryloxy group, a C7-C13 aralkyl group, a C7-C13 aralkoxy group, a C7-C13 alkylaryl group, a C7-C13 alkylaryloxy group, and a C2-C8 ether group. In one embodiment, at least one of R⁶, R⁷, R⁸, R⁹, and/or R¹⁰ group is a hydrogen.

In one embodiment, the organo-functional siloxane compound comprises an alkoxy group, an alkylaryl group, an ether group, or a combination of two or more thereof. Examples of suitable alkoxy groups include, but are not limited to, methoxy, ethoxy, propoxy, isopropoxy, butoxy, isobutoxy, etc. Examples of suitable alkylaryl groups include, but are not limited to, alkyl phenols. Examples of suitable ether groups include alkyl ethers such as, but not limited to, methyl ether groups, ethyl ether groups, propyl ether groups, butyl ether groups, etc., and combinations of two or more thereof.

In one embodiment, the organo-functional siloxane can be of the formula (11):

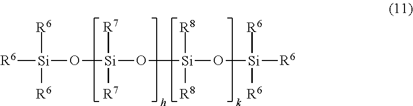

where R⁶, R⁷, R⁸, h, and k are described above. In one embodiment, the viscosity of the organo-functional silicon compound is from about 1 cSt at 25° C. to about 2,000 cSt at 25° C. In one embodiment, at least one of R⁶ is chosen from an alkyl, an aryl, alkoxy, an ether group, or combinations of two or more thereof.

In one embodiment, the organo-functional silicon compound is of the formula (12):

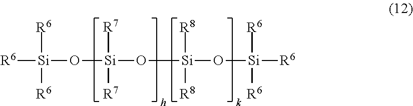

wherein h and k are described above and at least one R⁶, R⁷, or R⁸ is chosen from a group of the formula (13):

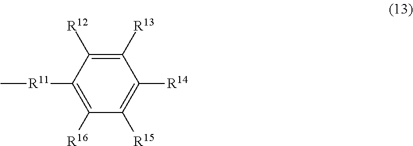

where R¹¹ is a divalent hydrocarbon and R¹², R¹³, R¹⁴, R¹⁵, and R¹⁶ are independently chosen from hydrogen, a hydroxy, an alkyl, a heteroalkyl, an alkoxy, an alkenyl, a heteroalkenyl, an alkenyloxy, a cycloalkyl, a heterocycloalkyl, a cycloalkoxy, an aryl, a heteroaryl, an aryloxy, an aralkyl, a heteroaralkyl, an alkylaryl, a heteroalkylaryl, an alkylaryloxy, an alkyl, aralkyl, alkylalkoxy, dialkoxy, heteroalkyl, heteroaryl, heteroaralkyl, or heteroalkylaryl bridge formed by one or more of R¹²-R¹³, R¹³-R¹⁴, R¹⁴-R¹⁵, and R¹⁵-R¹⁶, or a combination of two or more thereof.

In one embodiment, the organo-functional siloxane is alkyl-stopped. In one embodiment, the organo-functional siloxane is methyl-stopped. In one embodiment, the organo-functional siloxane is of the formula (14):

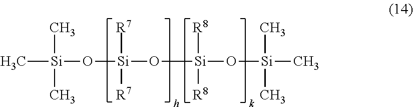

where R⁶, R⁷, R⁸, R⁹, h, and k are described above.

In one embodiment, the organo-functional siloxane is of the formula (15):

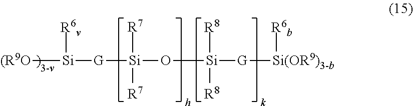

where v=0 or 1, b=0 or 1, G represents an oxygen atom or an unsubstituted bivalent hydrocarbon group, and R⁶, R⁷, R⁸, R⁹, h, and k are described above.

In one embodiment, the organo-functional siloxane comprises an alkylaryl group such as, for example an alkyl phenol group. In one embodiment, the organo-functional siloxane is of the formula:

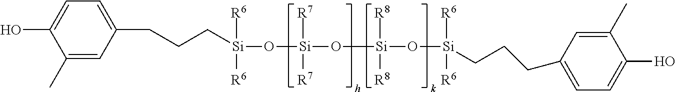

where R⁶, R⁷, R⁸, k, and k are described above.

In one embodiment, the organo-functional silicon compound is an organosilicon compound having hydrolyzable groups. Examples of suitable hydrolyzable groups include, but are not limited to an alkoxy group, an alkoxyalkoxy group, or a combination of two or more thereof. Non-limiting examples of suitable hydrolyzable groups include methoxy, ethoxy, propoxy, isopropoxy, butoxy, methoxyethoxy, etc., and combinations of two or more thereof. Still further examples of suitable organosilicon compounds include, but are not limited to, tetraethoxysilane, tetramethoxysilane, methyltrimethoxysilane, vinyltrimethoxysilane, methyltriethoxysilane, vinyltriethoxysilane, ethylorthosilicate, propylorthosilicate, partial hydrolysates of such compounds, and combinations of two or more thereof.

In one embodiment, at least one of the organo-functional silicon compound, a low-molecular-weight organic polymer, a high-boiling-point solvent, or a combination of two or more thereof has at least one hydridosilyl group, and the composition can be used to prepare a polymer by the dehydrogenative condensation reaction between a Si—OH group and a Si—H group to form Si—O—Si bonds and the release of hydrogen gas.

The organo-functional silicon material can be provided in an amount of from about 0.0001 to about 20 parts per weight per 100 parts per weight of the polymer component; 0.001 to 15 parts per weight; 0.01 to 10 parts per weight; even 0.1 to 5 parts per weight per 100 parts per weight of the polymer. Here as elsewhere in the specification and claims, numerical values can be combined to form new and non-disclosed ranges.

Additionally, the crosslinker and/or chain extender can be provided as part of a composition such as that disclosed in U.S. Patent Application Publication No. 2013/0303676, which is incorporated herein by reference in its entirety.

The extender can be provided in an amount of from about 0.0001 to about 20 parts per weight of the extender per 100 parts per weight of the polymer component; 0.001 to 15 parts per weight; 0.01 to 10 parts per weight; even 0.1 to 5 parts per weight per 100 parts per weight of the polymer. Here as elsewhere in the specification and claims, numerical values can be combined to form new and non-disclosed ranges.

In one embodiment, the crosslinker or chain extender (B) may be chosen from an alkoxysilane, an alkoxysiloxane, an oximosilane, an oximosiloxane, an enoxysilane, an enoxysiloxane, an aminosilane, a carboxysilane, a carboxysiloxane, an alkylamidosilane, an alkylamidosiloxane, an arylamidosilane, an arylamidosiloxane, an alkoxyaminosilane, an alkaryaminosiloxane, an alkoxycarbamatosilane, an alkoxycarbamatosiloxane, and combinations of two or more thereof.

Additional alkoxysilanes in an amount greater than 0.1 wt. % of component (A) that are not consumed by the reaction between the prepolymer Z′-X-Z′ and which comprise additional functional groups selected from R⁵ can also work as an adhesion promoter and are defined and counted under component (D).

The condensation catalyst (C) comprises an amine compound chosen from a secondary amine, a tertiary amine, a substituted amine, or a combination of two or more thereof. In embodiments, the amine may be chosen from a linear or cyclic aliphatic amine, an aromatic amine, a heterocyclic amine, an amino ester compound, or a combination of two or more thereof. The inventors have found that such compounds can accelerate the curing of compositions comprising compounds with a reactive silyl group. A secondary amine or tertiary amine may refer to amine compounds comprising hydrocarbon groups, which may be saturated or unsaturated. The term “substituted amine” as used herein refers to an amine comprising a group other than a hydrocarbon group attached to the amine nitrogen or a hydrocarbon group that is attached to an amine nitrogen.

In one embodiment, the catalyst is selected from a secondary amine, a tertiary amine, an aminosilane, or a combination of two or more thereof

In one embodiment, the catalyst comprises an aliphatic amine selected from a linear, a branched, a cyclic, a saturated, an unsaturated, a polyfunctional amine, or a combination of two or more thereof. The amine may comprise one or more other functional groups as part of the compound.

In one embodiment, the catalyst comprises an aromatic amine where the amine functionality is directly attached to the aromatic ring, attached via spacers, incorporated into the ring, or a combination of two or more thereof.

In one embodiment, the catalyst comprises one amine functional group or a plurality of amine functional groups. the amine compound comprises one or multiple amine functional group of the formula:

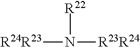

where R²² is independently chosen from hydrogen; a C₁-C₁₅ linear, branched, or cyclic alkyl group; a C₁-C₁₅ linear, branched, or cyclic alkyl group comprising one or more substituents chosen from a halide, N, O or S; a C₆-C₁₀ aryl group; a C₇-C₁₆ linear or branched alkylaryl group; a C₂-C₄ polyalkylene ether; or a linear or branched C₇-C₁₆ heteroaralkyl, heteroalkyl, heterocycloalkyl, or heteroaryl; and where R²³ and R²⁴ are independently chosen from a C₁-C₁₅ linear, branched, or cyclic alkyl group; a C₁-C₁₅ linear, branched, or cyclic alkyl group comprising one or more substituents chosen from a halide, N, O or S; a C₆-C₁₀ aryl group; a C₇-C¹⁶ linear or branched alkylaryl group; a C₂-C₄ polyalkylene ether; a linear or branched C₇-C₁₆ heteroaralkyl; heteroalkyl, heterocycloalkyl, heteroaryl, with the proviso that the nitrogen atom is bi-substituted with either of R²³R²⁴, R²³ and R²⁴, or a combined R²³, R²⁴ in the compound. That is, the nitrogen may be substituted with two R²³ groups, a R²³ and R²⁴ group, two R²⁴ groups, a R²³ group and a R²³R²⁴ group, a R²⁴ and a R²³R²⁴ groups, two R²³R²⁴ groups, etc.

In one embodiment, the catalyst comprises a secondary amine selected from dialkyl and substituted dialkyl amines, dimethylamine, diisopropylamine, dibutylamine, N-methylbutylamine, N,N-diallyl trimethylenediamine, diamylamine, dihexylamine, dioctylamine, N-ethylcetylamine, didodecylamine, ditetradecylamine, diricinoleylamine, N-isopropylstearylamine, N-isoamylhexylamine, N-ethyloctylamine, dioctadecylamine, their homologs and analogs, or a combination of two or more thereof.

In one embodiment, the catalyst comprises a secondary cycloalkylamine selected from dicyclohexylamine, N-methylcyclohexylamine, dicyclopentylamine, N-octylcyclohexylamine, N-octyl-3,5,5-trimethylcyclohexylamine, and their homologs and analogs; and unsaturated secondary amines, such as diallylamine, N-ethylallylamine, N-octylallylamine, dioleylamine, N-isopropylolelyamine, N-methyl-3,3,5-trimethyl-5-cyclohexenylamine, N-amyl-linoleylamine, N-methyl-propargylamine, diphenylamine, their analogs and homologs, or a combination of two or more thereof.

In one embodiment, the catalyst comprises a tertiary amine selected from trimethylamine, triethylamine, tri-isopropylamine, tributylamine, N-ethyldibutylamine, N-ethyl-N-butylamylamine, N,N-diethyl aniline, triallylamine, N,N-dipropylcyclohexylamine, N,N-dipropyloleyl-amine, trimethylamine, N- octyldiallylamine, N,N-dipropylcyclohexylamine, dimethylaminopropylemine, dimethylaminoethoxypropylamine, pentamethyldiethylylenetriamine, N-methylmorpholine, N-ethylmorpholine, N,N-dimethylbenzylamine, N,N-dimethylethanolamine, N,N,N′,N′-tetramethyl-1,4-butanediamine, N,N-dimethylpiperazine, bis(dimethylaminoethyl)ether, bis(2-dimethylaminoethyl)ether, morpholine, N-substituted morpholines, such as N-methyl or N-ethyl morpholine, 4,4′-(oxydi-2,1-ethanediyl)bis, triethylenediamine, pentamethyl diethylene triamine, dimethyl cyclohexyl amine, N-cetyl N,N-dimethyl amine, N-coco-morpholine, N,N-dimethyl aminomethyl N-methyl ethanol amine, N,N,N′-trimethyl-N′-hydroxyethyl bis(aminoethyl)ether, N,N-bis(3-dimethylaminopropyl)N-isopropanolamine, (N,N-dimethyl)amino-ethoxy ethanol, N,N,N′,N′-tetramethyl hexane diamine, N,N-dimorpholinodiethyl ether, N-methyl imidazole, dimethyl aminopropyl dipropanolamine, bis(dimethylaminopropyl)amino-2-propanol, tetramethylamino bis (propylamine), (dimethyl(aminoethoxyethyl))((dimethyl amine)ethyl)ether, tris(dimethylamino propyl) amine, dicyclohexyl methyl amine, bis(N,N-dimethyl-3-aminopropyl) amine, N,N-bis (3-dimethylaminopropyl)-N-isopropanolamine, 1,3-propanediamine, 1,2-ethylene piperidine, methyl-hydroxyethyl piperazine, dimethylaminopropyl-S-triazine, bisdimethylaminopropylurea, their analogs and homologs, or a combination of two or more thereof.

In one embodiment, the catalyst comprises a heterocyclic amine selected from piperidine, pyridine, methylpiperazine, 2,2,4,6-tetramethylpiperidine, 2,2,4,6-tetramethyl-tetrahydropyridine, N-ethyl 2,2,4,6 tetramethylpiperidine, 2-aminopyrimidine, 2-aminopyridine, 2-(dimethylamino)pyridine, 4-(dimethylamino)pyridine, 2-hydroxypyridine, imidazole, 2-ethyl-4-methylimidazole, morpholine, N-methylmorpholine, piperidine, 2-piperidinemethanol, 2-(2-piperidino)ethanol, piperidone, 1,2-dimethyl- 1,4,5,6-tetrahydropyrimidine, aziridine, methoymethyldiphenylamine, nicotine, pentobarbital, methadone, cocaine, and triphenylamine, or a combination of two or more thereof.

In one embodiment, the catalyst is selected from diethanolamine, triethanolamine, N-methyl-1,3-propanediamine, N,N′-dimethyl-1,3-propanediamine, diethylenetriamine, triethylenetetramine, 2-(2-aminoethylamino)ethanol, 3-dimethylaminopropylamine, 3-diethylaminopropylamine, 3-dibutylaminopropylamine, 3-morpholinopropylamine, 2-(1-piperidinyl)ethylamine, and 2,4,6-tris(dimethylaminomethyl)phenol, or a combination of two or more thereof.

In one embodiment, the catalyst (C) comprises an amino ester compound comprising at least one amino ester functional group. In one embodiment, the amino ester compound comprises a plurality of amino ester functional groups. The number of amino ester functional groups is not particularly limited, and can be chosen as desired for a particular purpose or intended application. The activity of the amino ester compound as a catalyst has been found to increase with a greater number of amino ester functional groups. In one embodiment, the amino ester compound comprises one or more amino ester functional groups; three or more amino ester functional groups; four or more amino ester functional groups; even five or more amino ester functional groups. In one embodiment, the amino ester compound comprises 1-10 amino ester functional groups; 2-8 amino ester functional groups; even 3-6 amino ester functional groups.

The amino ester compound may be a beta-amino ester compound. The amino ester functional group in the amino ester compound can be of the formula:

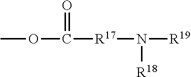

where R¹⁷ is a C₁-C₅ alkyl group, and R¹⁸ and R¹⁹ are independently chosen from hydrogen, a C₁-C₁₀ linear, branched, or cyclic alkyl group, a C₁-C₁₀ linear, branched, or cyclic alkyl group comprising one or more substituents chosen from a halide, N, O or S; a C₆-C₁₀ aryl group; a C₇-C₁₆ alkylaryl group; a C₇-C₁₆ arylalkyl group; a C₂-C₄ polyalkylene ether; a substituted silicon, a substituted siloxane, or a combination of two or more thereof. Non-limiting examples of suitable groups for the R¹⁸ and R¹⁹ groups include, hydrogen, a C₁-C₁₀ alkyl group such as methyl, ethyl, propyl, isopropyl, butyl, isobutyl, etc., a substituted C₁-C₁₀ alkyl such as an alkyl ether, a hydroxyl terminated alkyl group, an amine terminated alkyl group, etc., and an alkyl alkoxy silane group. In one embodiment, R¹⁸ is hydrogen, and R¹⁹ is chosen from a C₁-C₅ alkyl, or an alkyl alkoxy silane of the formula:

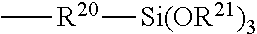

where R²⁰ and R²¹ are independently chose from a C₁-C₁₀ alkyl.

Non-limiting examples of suitable R¹⁹ groups include:

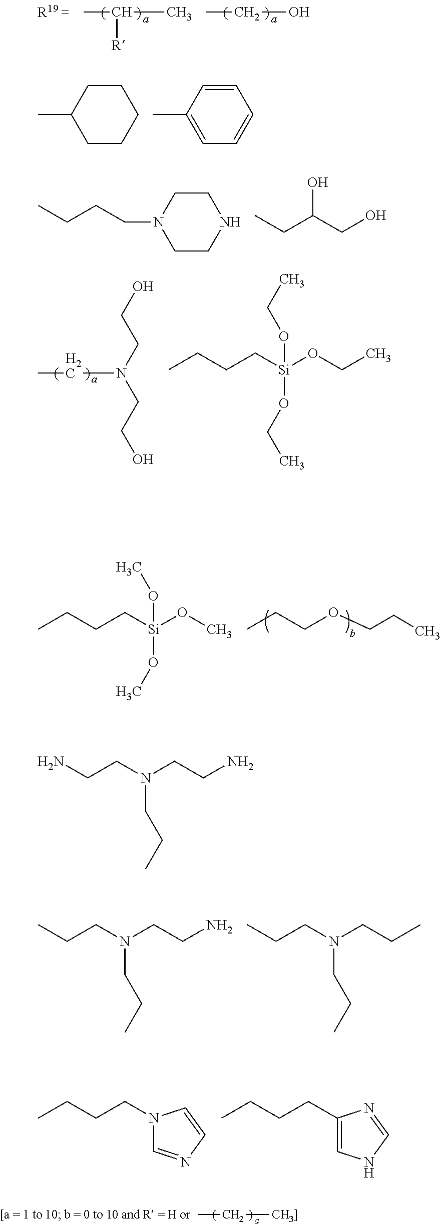

The amino ester can be symmetrical or unsymmetrical. It may comprise saturated or unsaturated groups. The amino ester compound can, in one embodiment, be a compound of the formula:

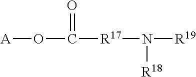

where A is chosen from hydrogen, a C₁-C₁₀ linear, branched, or cyclic alkyl group, a C₁-C₁₀ linear, branched, or cyclic alkyl group comprising one or more substituents chosen from a halide, N, O or S; a C₆-C₁₀ aryl group; a C₇-C₁₆ alkylaryl group; a C₇-C₁₆ arylalkyl group; a C₂-C₄ polyalkylene ether; a substituted silicon, a substituted siloxane, or:

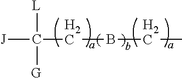

where B is a C₁-C₁₀ linear, branched, or cyclic alkyl group, a C₁-C₁₀ linear, branched, or cyclic alkyl group comprising one or more substituents chosen from a halide, N, O or S; a C₆-C₁₀ aryl group; or a silicon containing compound of the formula [R¹_{c}R²_{3-c}Si-Z-]ₙX-Z-SiR¹_{c}R²_{3-c}, where R¹, R², Z, X, and c can be as described above; and G, J, and L are independently chosen from hydrogen and an amino ester group of the formula:

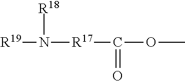

where R¹⁷, R¹⁸, and R¹⁹ can be as described above; a is 1 to 10; b is 0 to 10.

In one embodiment, B is a silicon containing unit such as, for example, a unit of the formula [R¹_{c}R²_{3-c}Si-Z-]ₙX-Z-SiR¹_{c}R²_{3-c}. In one embodiment, the silicon containing unit is an alkyl siloxane unit. Non-limiting example of suitable alkyl siloxanes include methyl siloxane, ethyl siloxane, etc.

In one embodiment, A is of the formula:

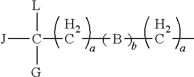

and at least one of G, J, and L is chosen from an amino ester group of the formula

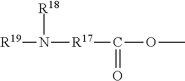

where R¹⁷, R¹⁸, and R¹⁹ can be described as above.

Non-limiting examples of suitable amino esters include:

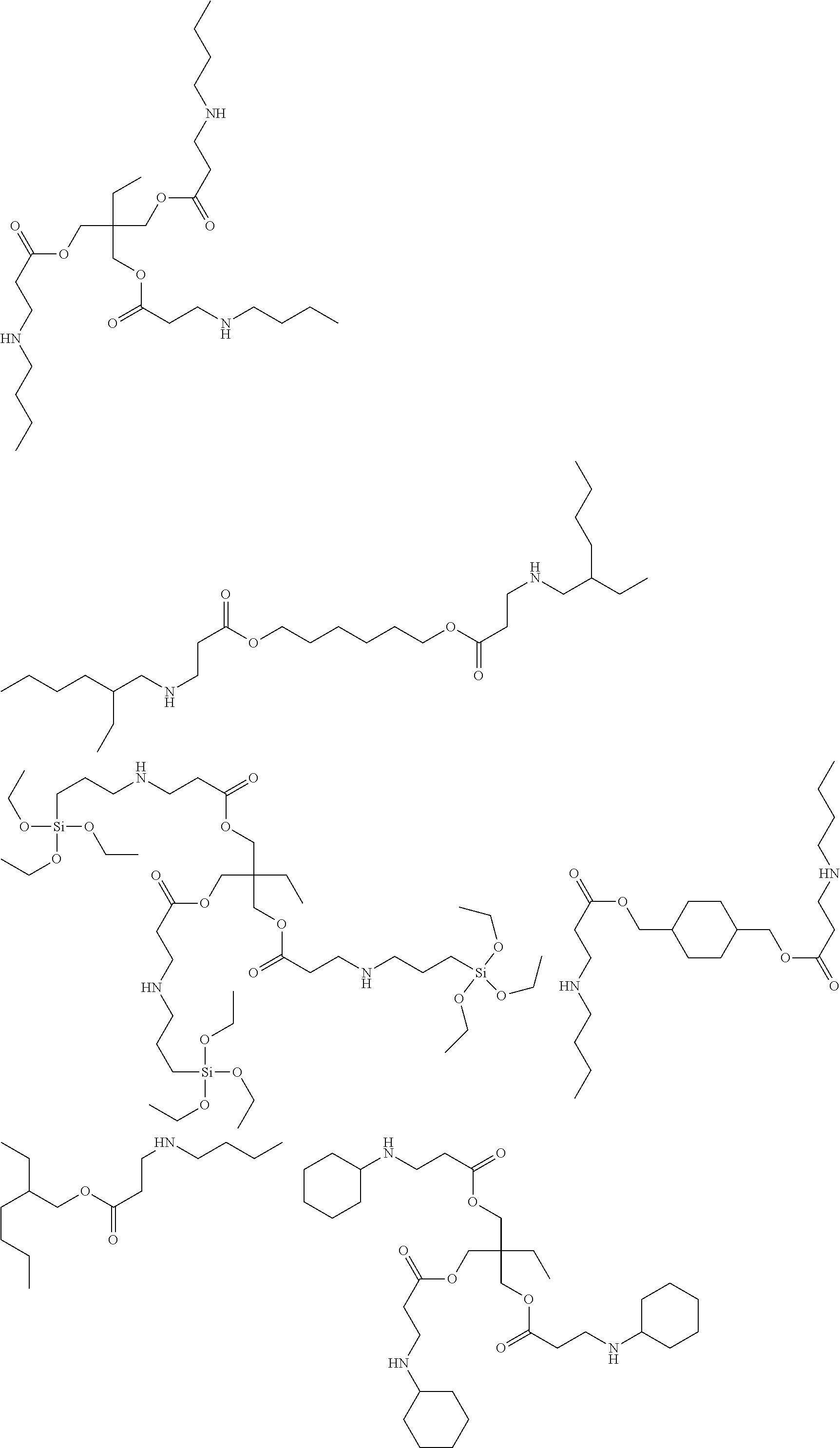

In still other embodiments, the amino ester can be a poly amino ester comprising a plurality of repeat amino ester functional groups. The poly amino ester can have molecular weight of the range of 50 g/mol to 10000 g/mol; 100 g/mol to 5000 g/mol; 250 g/mol to 2500 g/mol; even about 500 g/mol to about 1000 g/mol. In another embodiment, the polymer derived from the amioester in the present invention have a pKa in the range 5.5 to 8.5. Further polymer may be designed to have a desired pKa between 3.0 to 9.0. In certain embodiments, the polymer has more than one acidic and or basic moiety resulting in more than one pKa. The present invention also provides methods of making beta-amino esters suitable for use as the catalyst component (C). Beta-amino esters can be synthesized through a Michael addition reaction of an acrylate and an amine. The reaction is carried out at room temperature without the need for any catalyst. The reaction is generally free of any by-product. The desired amino ester can be formed by choosing appropriate functional acrylate and amine compounds to conduct the reaction.

The catalyst component (C) comprising the amino ester can be present in an amount of from 0.0001 to about 10 parts per weight (wt. pt.) based on 100 parts off the polymer (A); 0.005 to about 7.5 wt. pt. based on 100 parts of the polymer (A); about 0.01 to about 5.0 wt. pt. based on 100 parts of the polymer component (A); from about 0.15 to about 2.0 wt. pt. based on 100 parts of the polymer component (A); even from about 0.5 to about 1.5 wt. pt. of the polymer component (A). In one embodiment, the catalyst (C) is present in an amount of from about 0.01 to about 1 wt. pt. based on 100 parts of the polymer (A); from about 0.025 to about 0.8 wt. pt. based on 100 parts of the polymer (A); even from about 0.05 to about 0.5 wt. pt. based on 100 parts of the polymer (A).

The composition optionally includes an adhesion promoter component (D) that is different from component (A) or (B). In another embodiment, the curable compositions comprise an adhesion promoter. The amino esters can be used with a wide range of adhesion promoters.

In one embodiment, the adhesion promoter (D) may be an organofunctional silane comprising the group R⁵, e.g., aminosilanes, and other silanes that are not identical to the silanes of component (B), or are present in an amount that exceeds the amount of silanes necessary for endcapping the polymer (A). The amount of non-reacted silane (B) or (D) in the reaction for making (A) can be defined in that after the endcapping reaction the free silanes are evaporated at a higher temperature up to 200° C. and vacuum up to 1 mbar to be more than 0.1 wt. % of (A).

Thus, some selected amines can advantageously be added to fine tune the rate of the metal-complex-catalyzed condensation curing of silicone/non-silicone polymer containing reactive silyl groups, as desired.

In one embodiment, the composition comprises an adhesion promoter (D) comprising a group R⁵ as described by the general formula (16):

R⁵_{g}R¹_{d}Si(R²)_{4-d-g} (16)

where R⁵ is E-(CR³₂)ₕ-W-(CH₂)ₕ-; R¹, R², and d are as described above; g is 1 or 2; d+g=1 to 2; and h is 0 to 8, and may be identical or different.

Non-limiting examples of suitable compounds include:

E¹-(CR³₂)ₕ-W-(CH₂)ₕ-SiR¹_{d}(R²)_{3-d} (16a) or (16d)

E²-[(CR³₂)ₕ-W-(CH₂)ₕ-SiR¹_{d}(R²)_{3-d}]ⱼ (16b) or (16f)

where j is 2 to 3.

The group E may be selected from either a group E¹ or E². E¹ may be selected from a monovalent group comprising amine, —NH₂, —NHR, —(NHC₂H₅)ₐNHR, NHC₆H₅, halogen, pseudohalogen, unsaturated aliphatic group with up to 14 carbon atoms, epoxy-group-containing aliphatic group with up to 14 carbon atoms, cyanurate-containing group, and an isocyanurate-containing group.

E² may be selected from a group comprising a di- or multivalent group consisting of amine, polyamine, cyanurate-containing, and an isocyanurate-containing group, sulfide, sulfate, phosphate, phosphite, and a polyorganosiloxane group, which can contain R⁵ and R² groups; W is selected from the group consisting of a single bond, a heteroatomic group selected from —COO—, —O—, epoxy, —S—, —CONH—, —HN—CO—NH— units; R³ is as defined above, R¹ may be identical or different as defined above, R² is defined as above and may be identical or different.

Non-limiting examples of component (D) include:

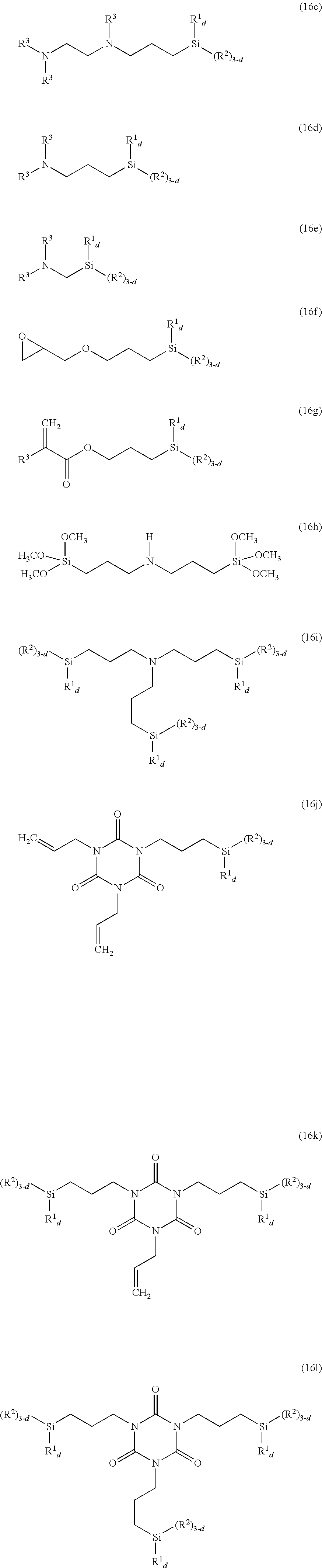

wherein R¹, R², and d are as defined above. Examples of component (D) include compounds of the formulas (16a-16l). Furthermore the formula (16b) of compounds (D) shall comprise compounds of the formula (16m):

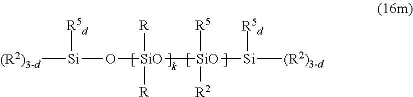

wherein: R, R², R⁵, and d are as defined above; k is 0 to 6 (and in one embodiment desirably 0); b is as described above (in one embodiment desirably 0 to 5); and 1+b≦10. In one embodiment, R⁵ is selected from:

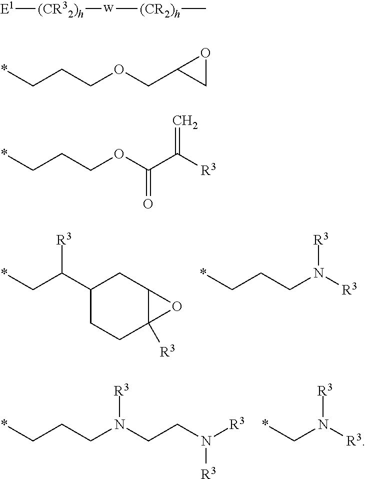

An exemplary group of adhesion promoters are selected from the group that consists of amino-group-containing silane coupling agents. The amino-group-containing silane adhesion promoter agent (D) is an acidic compound having a group containing a silicon atom bonded to a hydrolyzable group (hereinafter referred to as a hydrolyzable group attached to the silicon atom) and an amino group. Specific examples thereof include the same silyl groups with hydrolyzable groups described above. Among these groups, the methoxy group and ethoxy group are particularly suitable. The number of the hydrolyzable groups may be 2 or more, and particularly suitable are compounds having 3 or more hydrolyzable groups.

Examples of other suitable adhesion promoter (D) include, but are not limited to N-(2-aminoethyl)aminopropyltrimethoxysilane, gamma-aminopropyltriethoxysilane, gamma-aminopropyltrimethoxysilane, bis(3-trimethoxysilypropyl)amine, N-phenyl-gamma-aminopropyltrimethoxysilane, triaminofunctionaltrimethoxysilane, gamma-aminopropylmethyldimethoxysilane, gamma-aminopropylmethyldiethoxysilane, methacryloxypropyltrimethoxysilane, methylaminopropyltrimethoxysilane, gamma-glycidoxypropylethyldimethoxysilane, gamma-glycidoxypropyltrimethoxysilane, gamma-glycidoxyethyltrimethoxysilane, gamma-glycidoxypropylmethyldimethoxysilane, gamma-glycidoxypropylmethyldiethoxysilane, beta-(3,4-epoxycyclohexyl)ethyltrimethoxysilane, beta-(3,4-epoxycyclohexyl)ethylmethyldimethoxysilane, beta-(3,4-epoxycyclohexyl)ethyltriethoxysilane, beta-(3,4-epoxycyclohexyl)ethylmethyldiethoxysilane, epoxylimonyltrimethoxysilane, isocyanatopropyltriethoxysilane, isocyanatopropyltrimethoxysilane, isocyanatopropylmethyldimethoxysilane, beta-cyanoethyltrimethoxysilane, gamma-acryloxypropyltrimethoxysilane, gamma-methacryloxypropylmethyldimethoxysilane, alpha, omega-bis(aminoalkyldiethoxysilyl)polydimethylsiloxanes (Pn=1-7), alpha, omega-bis(aminoalkyldiethoxysilyl)octamethyltetrasoxane, 4-amino-3,3-dimethylbutyltrimethoxysilane, and N-ethyl-3-trimethoxysilyl-2-methylpropanamine, 3-(N,N-diethylaminopropyl) trimethoxysilane combinations of two or more thereof, and the like. Particularly suitable adhesion promoters include bis(alkyltrialkoxysilyl)amines and tris(alkyltrialkoxysilyl)amines including, but not limited to, bis(3-trimethoxysilylpropyl)amine and tris(3-trimethoxysilylpropyl)amine.

Also it is possible to use derivatives obtained by modifying them, for example, amino-modified silyl polymer, silylated amino polymer, unsaturated aminosilane complex, phenylamino long-chain alkyl silane and aminosilylated silicone. These amino-group-containing silane coupling agents may be used alone, or two or more kinds of them may be used in combination.

The adhesion promoter (D) may be present in an amount of from about 0.1 to about 5.0 wt. % based on 100 parts of the polymer component (A). In one embodiment, the adhesion promoter may be present in an amount of from about 0.15 to about 2.0 wt. % based on 100 parts of the polymer component (A). In another embodiment, the adhesion promoter may be present in an amount of from about 0.5 to about 1.5 wt. % of the polymer component (A). This defines the amount of (D) in composition of (A) wherein the content of free silanes coming from the endcapping of polymer (A) is smaller than 0.1 wt. %.

The present compositions may further include a filler component (E). The filler component(s) (E) may have different functions, such as to be used as reinforcing or semi-reinforcing filler, i.e., to achieve higher tensile strength after curing. The filler component may also have the ability to increase viscosity, establish pseudoplasticity/shear thinning, and demonstrate thixotropic behavior. Non-reinforcing fillers may act as volume extenders. The reinforcing fillers are characterized by having a specific surface area of more than 50 m²/g related BET-surface, whereby the semi-reinforcing fillers have a specific surface area in the range of 10-50 m²/g. So-called extending fillers have preferably a specific surface area of less than 10 m²/g according to the BET-method and an average particle diameter below 100 μm. In one embodiment, the semi-reinforcing filler is a calcium carbonate filler, a silica filler, or a mixture thereof. Examples of suitable reinforcing fillers include, but are not limited to, fumed silicas or precipitated silicas, which can be partially or completely treated with organosilanes or siloxanes to make them less hydrophilic and decrease the water content or control the viscosity and storage stability of the composition. These fillers are named hydrophobic fillers. Tradenames are Aerosil®, HDK®, Cab-O-Sil® etc.

Examples of suitable extending fillers include, but are not limited to, ground silicas (Celite™), precipitated and colloidal calcium carbonates (which are optionally treated with compounds such as stearate or stearic acid); reinforcing silicas such as fumed silicas, precipitated silicas, silica gels and hydrophobized silicas and silica gels; crushed and ground quartz, cristobalite, alumina, aluminum hydroxide, titanium dioxide, zinc oxide, diatomaceous earth, iron oxide, carbon black, powdered thermoplastics such as acrylonitrile, polyethylene, polypropylene, polytetrafluoroethylene and graphite or clays such as kaolin, bentonite or montmorillonite (treated/untreated), and the like.

The type and amount of filler added depends upon the desired physical properties for the cured silicone/non-silicone composition. As such, the filler may be a single species or a mixture of two or more species. The extending fillers can be present from about 0 to about 300 wt. % of the composition related to 100 parts of component (A). The reinforcing fillers can be present from about 5 to about 60 wt. % of the composition related to 100 parts of component (A), preferably 5 to 30 wt. %.

The inventive compositions optionally comprise an acidic compound (F), which, in conjunction with the adhesion promoter and amino ester, may accelerate curing (as compared to curing in the absence of such compounds). The component (F) may be present in an amount of from about 0.01 to about 5 wt. % of the composition. In another embodiment 0.01 to about 8 parts per weight (pt. wt.) per 100 pt. wt. of component (A) are used, more preferably 0.02 to 3 pt. wt. per 100 pt. wt. of component (A) and most preferably 0.02 to 1 pt. wt. per 100 pt. wt. of component (A) are used.

The acidic compounds (F) may be chosen from various phosphate esters, phosphonates, phosphites, phosphonites, sulfites, sulfates, pseudohalogenides, branched alkyl carboxylic acids, combinations of two or more thereof, and the like. Without being bound to any particular theory, the acidic compounds (F) may, in one embodiment, be useful as stabilizers in order to ensure a longer storage time when sealed in a cartridge before use in contact with ambient air. Especially alkoxy-terminated polysiloxanes can lose the ability to cure after storage in a cartridge and show decreased hardness under curing conditions. It may, therefore be useful to add compounds of the formula (9), which can extend storage time or ability to cure over months.

O═P(OR⁶)_{3-c}(OH)_{c} (9)

whereby c is as defined above; and R⁶ is selected from the group of linear or branched and optionally substituted C₁-C₃₀ alkyl groups, linear or branched C₅-C₁₄ cycloalkyl groups, C₆-C₁₄ aryl groups, C₆-C₃₁ alkylaryl groups, linear or branched C₂-C₃₀ alkenyl groups or linear or branched C₁-C₃₀ alkoxyalkyl groups, C₄-C₃₀₀ polyalkenylene oxide groups (polyethers), such as Marlophor® N5 acid, triorganylsilyl- and diorganyl (C₁-C₈)-alkoxysilyl groups. The phosphates can include also mixtures of primary and secondary esters. Non-limiting examples of suitable phosphonates include 1-hydroxyethane-(1,1-diphosphonic acid) (HEDP), aminotris(methylene phosphonic acid) (ATMP), diethylenetriaminepenta(methylene phosphonic acid) (DTPMP), 1,2-diaminoethane-tetra(methylene phosphonic acid) (EDTMP), and phosphonobutanetricarboxylic acid (PBTC).

In another embodiment, a compound of the formula O═P(OR⁷)_{3-g}(OH)_{g} may be present or added where g is 1 or 2, and R⁷ is defined as R⁶ or di- or mulitvalent hydrocarbons with one or more amino group.

Another type are phosphonic acid compounds of the formula R⁶P(O)(OH)₂ such as alkyl phosphonic acids preferably hexyl or octyl phosphonic acid.

In one embodiment, the acidic compound may be chosen from a mono ester of phosphoric acid of the formula (R⁸O)PO(OH)₂; a phosphonic acid of the formula R⁸P(O)(OH)₂; or a monoester of phosphorous acid of the formula (R⁸O)P(OH)₂ where R⁸ is a C₁-C₁₈ alkyl, a C₂-C₂₀ alkoxyalkyl, phenyl, a C₇-C₁₂ alkylaryl, a C₂-C₄ polyalkylene oxide ester or its mixtures with diesters, etc.

In another embodiment, the acidic compound is a carboxylic acid, including, for example, a C₄-C₃₀ carboxylic acid, a branched C₄-C₃₀ alkyl carboxylic acids, including C₅-C₁₉ acids with an alpha tertiary carbon, or a combination of two or more thereof. Examples of such suitable compounds include, but are not limited to, Versatic™ Acid, lauric acid, and stearic acid. In one embodiment, the acidic compound may be a mixture comprising branched alkyl carboxylic acids. In one embodiment, the acidic compound is a mixture of mainly tertiary aliphatic C₁₀ carboxylic acids.

Generally, the acidic component (F) is added in a molar ratio of less than or equal to 1 with respect to catalyst (C). In embodiments, the acidic component (F) is added in a molar ratio of (F):(C) of 1:15 to 1:1.

The curable composition may also include auxiliary substances (G) such as plastizers, pigments, stabilizers, anti-microbial agents, fungicides, biocides, and/or solvents. Preferred plastizers for reactive polyorganosiloxanes (A) are selected from the group of polyorganosiloxanes having chain lengths of 10 to 300 siloxy units. Preferred are trimethylsilyl terminated polydimethylsiloxanes having a viscosity of 100 to 1000 mPa·s at 25° C. The choice of optional solvents (dispersion media or extenders) may have a role in assuring uniform dispersion of the accelerator, thereby altering curing speed. Such solvents include polar and non-polar solvents such as toluene, hexane, chloroform, methanol, ethanol, isopropyl alcohol, acetone, methylethyl ketone, dimethylformguanidine-containing (DMF), dimethyl sulfoxide (DMSO), N-methylpyrrolidinone (NMP), and propylene carbonate. Water can be an additional component (G) to accelerate fast curing 2-part compositions RTV-2, whereby the water can be in one part of the two compositions. Particularly suitable non-polar solvents include, but are not limited to, toluene, hexane, and the like if the solvents should evaporate after cure and application. In another embodiment, the solvents include high-boiling hydrocarbons such as alkylbenzenes, phthalic acid esters, arylsulfonic acid esters, trialkyl- or triarylphosphate esters, which have a low vapor pressure and can extend the volume providing lower costs. Examples cited by reference may be those of U.S. Pat. Nos. 6,599,633; 4,312,801. The solvent can be present in an amount of from about 20 to about 99 wt. % of the catalyst composition.

Applicants have found that using amino esters as a catalyst may provide a curable composition that yields a cured polymer exhibiting suitable tack-free time, hardness, and/or cure time, and may even be comparable to compositions made using tin catalysts. The curing properties can be controlled by using the amino ester catalyst with one or more adhesion promoters.

In one embodiment, a composition in accordance with the present invention comprises: 100 wt. % polymer component (A); about 0.1 to about 10 wt. % crosslinker component (B); and about 0.01 to about 7 wt. % catalyst (C). In one embodiment, the composition further comprises from about 0.1 to about 5 wt. %, in one embodiment 0.15 to 1 wt. %, of an adhesion promoter component (D); about 0 to about 300 pt. wt. filler component (E); about 0.01 to about 7 wt. % of acidic compound (F); optionally 0 to about 15 wt. % component (G), where the wt. % of components (B)-(G) are each based on 100 parts of the polymer component (A). In one embodiment, the composition comprises the component (F) in an amount of from about 0.01 to about 1 wt. % per 100 pt. wt. of component (A). In still another embodiment, the composition comprises the accelerator (C) in an amount of from about 0.1 to about 0.8 wt. % per 100 wt. % of component (A).

It will be appreciated that the curable compositions may be provided as either a one-part composition or a two-part composition. A one-part composition refers to a composition comprising a mixture of the various components described above. A two-part composition may comprise a first portion and a second portion that are separately stored and subsequently mixed together just prior to application for curing. In one embodiment, a two-part composition comprises a first portion (P1) comprising a polymer component (A) and a crosslinker component (B), and a second portion (P2) comprising the catalyst component (C) comprising the amino ester. The first and second portions may include other components (F) and/or (G) as may be desired for a particular purpose or intended use. In one embodiment, the first portion (P1) may optionally comprise an adhesion promoter (D) and/or a filler (E), and the second portion (P2) may optionally comprise auxiliary substances (G), a cure rate modifying component (F), and water.

In one embodiment, a two-part composition comprises (i) a first portion comprising the polymer component (A), optionally the filler component (E), and optionally the acidic compound (F); and (ii) a second portion comprising the crosslinker (B), the catalyst component (C), optionally the adhesive promoter (D), and optionally the acidic compound (F), where portions (i) and (ii) are stored separately until applied for curing by mixing of the components (i) and (ii).

An exemplary two-part composition comprises: a first portion (i) comprising 100 pt. wt. of component (A), and 0 to 70 pt. wt. of component (E); and a second portion (ii) comprising 0.1 to 5 pt. wt. of at least one crosslinker (B); 0.01 to 4 pt. wt. of a catalyst (C); 0.1 to 2 pt. wt. of an adhesion promoter (D); and 0.02 to 1 pt. wt. component (F).

All these polymerization/crosslinking routes result in more or less polymerized and more or less crosslinked silicone products which can constitute products that can be used in multiple applications. The curable compositions may be used in a wide range of applications including as materials for sealing, mold making, glazing, prototyping; as adhesives; as coatings in sanitary rooms; as joint seal between different materials, e.g., sealants between ceramic or mineral surfaces and thermoplastics; as paper release; as impregnation materials; weather strip coatings, release coatings, adhesives, adhesion finishes, leak tight products, pointing products, foams, etc. A curable composition in accordance with the present invention comprising an amino ester as an accelerator may be suitable for a wide variety of applications such as, for example, a general purpose and industrial sealant, potting compound, caulk, adhesive or coating for construction use, insulated glass, structural glazing, where glass sheets are fixed and sealed in metal frame; caulks, adhesives for metal plates, car bodies, vehicles, electronic devices, and the like. Furthermore, the present composition may be used either as a one-part RTV-1 or as a two-part RTV-2 formulation that can adhere onto broad variety of metal, mineral, ceramic, rubber, or plastic surfaces.

Curable compositions comprising amino ester catalysts with or without organic additives as cure accelerators may be further understood with reference to the following Examples.

### EXAMPLES

### General Experimental Procedure

The results presented below are prepared using formulation with two different types of component A, where in silanol-stopped PDMS+silica filler+low molecular weight PDMS and polysilicate is premixed and used as component A1, The other type of formulation prepared by mixing silanol-stopped PDMS+silica filler+low molecular weight PDMS) will be referred hence forth component A2 is designed for to achieve the faster curability and good adhesion to different substrates such as glass, aluminum, and plastic substrates.

A mixture was created with approximately 1 gram of ethyl polysilicate (EPS)), 0.6 to 1.4 grams of mixture of amino-functional silanes, 0.1 to 0.5 grams of amino ester catalyst, and approximately 97 to 99.5 grams of mixture of (silanol-stopped PDMS+silica filler+low molecular weight PDMS). The mixture was mixed using a Hauschild mixer for 1.5 min. The mixed formulation was poured into a Teflon mold (L×W×D =10 cm×10 cm×1 cm) and placed inside a fume hood. The surface curing (TFT) and bulk curing was monitored as a function of time (maximum of 3 days).

### Heat Ageing Method

The premixed mixture containing ethyl polysilicate (adhesion promoter/s, organo-functional siloxane/s and catalyst were kept in an oven for (1) 4 hours at 50° C., or (2) 5 days at 70° C. After the specified period, the mixture was removed from the oven and allowed to return to ambient temperature. This mixture was then combined with component A and mixed on a Hauschild mixer for 1.5 min. The mixed formulation was poured into a Teflon mold (L×W×D=10 cm×10 cm×2 cm) and placed inside a fume hood. These heat-ageing procedures should simulate the storage effect at room temperature over longer time periods.

### Tack-Free Time (TFT) Measurement Method A

In a typical TFT measurement, the premixed composition of component A and component B is poured into a Teflon mold (L×W×D=50 mm×30 mm×20 mm) and spread evenly using a stainless steel spatula. A 10-gram, stainless steel weight/stainless steel spatula was placed on the surface of the formulation to determine the tackiness of the surface. TFT is defined as the time taken for getting a non-tacky surface. This time is recorded to the nearest minute.

### Tack-Free Time Measurement Method B

Tack-free time was determined using finger soft touch method wherein the dried finger is softly placed on the surface of formulation and checked for non-sticky surface and recorded.

### Shore A Hardness Measurement Method

Shore A hardness values were determined by preparing three samples of dimension (50 mm×30 mm×10 mm/20 mm). The sample specimens were taken out of the mold after the interval of 24 hrs. (samples-1), 48 hrs. (Sample-2) and 72 hrs. (Sample-3). The shore A measurement was performed both on top and bottom immediately after taking it out from the mold. This measurement method was used as a measure of time required for bulk cure of the sample. Bulk cure time is the time required for complete curing of formulation throughout the thickness (i.e. top to bottom).

### Substrate Adhesion Test Method

Cohesive failure to glass, metal, and plastic substrates was determined in the following manner. The premixed composition of component A and component B was applied as thick lines on the pre-cleaned and dried standard plastic, glass and metal substrates. The substrates were kept at room temperature for three days. After three days, the adhered and cured materials were removed from substrates to check the cohesive or adhesive failure.

TABLE 1
Comparison of TFT and Bulk Cure Properties

| | C1 | 1 | 2 | 3 | 4 |
|---|---|---|---|---|---|
| Component A1 | 100 | 100 | | | |
| Component A2 | | | 100 | 100 | 100 |
| EPS | | | 1 | 1 | 1 |
| bis-[gamma- | | | 0.8 | 0.8 | 0.8 |
| (trimethoxysilyl)propyl]amine | | | | | |
| N-(beta-aminoethyl)-gamma- | | | 0.6 | 0.6 | 0.6 |
| aminopropyl trimethoxysilane | | | | | |
| DBTDL (dibutyl tin dilaurate) | 0.5 | | | | |
| Dicyclohexylamine | | 0.4 | | | |
| 2,2(ethylenedioxy) bis-ethylene | | | 0.4 | | |
| diamine | | | | | |
| N-propyl ethylene diamine | | | | 0.4 | |
| Methoxy stopped PDMS | | 0.1 | | | |
| N-(2-hydroxy)-ethylenediamine | | | | | 0.4 |

Curability

| | | | | | |
|---|---|---|---|---|---|
| TFT (min) | 173 | 97 | 41 | 34 | 38 |
| Hardness (3 d) | 50/49 | 45/45 | 32/32 | 31/29 | 29/27 |

Adhesion (3 d)

| | | | | | |
|---|---|---|---|---|---|
| ABS | NT | NT | x | ∘ | ∘ |
| PBT | NT | NT | x | ∘ | ∘ |
| AC | NT | NT | x | x | ∘ |

Test sample thickness = #10 mm
NT = not tested
Adhesion: ∘ = CF; x = adhesion failed

### Examples 5-20

Examples 5-20 were prepared by using Component Al. The compositions were cured as described above by mixing the different organic amines described in the Examples. Examples 5-11 are comparative examples using primary amines. Tables 1 and 2 show results for the curable compositions.

TABLE 2
Comparison of TFT and Hardness Properties

| | 5 | 6 | 7 | 8 | 9 | 10 | 11 | 12 | 13 |
|---|---|---|---|---|---|---|---|---|---|
| PDMS-OH | 100 | | | | | | | | |
| Silica & CaCO₃ | | | | | | | | | |
| Polycondensed TEOS | | | | | | | | | |
| Allyl amine | 1 | | 1 | | | | | | |
| Butyl amine | | 0.1 | | | | | | | |
| Decyl amine | | | | 1 | 0.5 | | | | |
| 2-ethylhexylamine | | | | | | 0.5 | 1 | | |
| Diethanolamine | | | | | | | | 1 | |
| Dicyclohexylamine | | | | | | | | | 1 |
| 1-methylpiperazine | | | | | | | | | |
| N (2-hydroxyethyl)ethylenediamine | | | | | | | | | |
| 2,2′(ethylenedioxy)bis(ethylamine) | | | | | | | | | |
| Morpholine | | | | | | | | | |
| Methylbenzylamine | | | | | | | | | |
| A-methylbenzylamine | | | | | | | | | |
| Tetraethylenepentamine | | | | | | | | | |
| TFT (min) | 18 | 23 | 18 | 6 | 8 | 14 | 7 | 46 | 47 |
| Hardness (Shore A) - 2 days | 33/44 | 33/43 | 33/44 | 40/37 | 40/42 | 43/43 | 42/42 | 28/39 | 44/43 |

| | | | | | | |
|---|---|---|---|---|---|---|
| 14 | 15 | 16 | 17 | 18 | 19 | 20 |

| | |
|---|---|
| PDMS-OH | 100 |

| Silica & CaCO₃ | | | | | | | |
|---|---|---|---|---|---|---|---|
| Polycondensed TEOS | | | | | | | |
| Allyl amine | | | | | | | |
| Butyl amine | | | | | | | |
| Decyl amine | | | | | | | |
| 2-ethylhexylamine | | | | | | | |
| Diethanolamine | | | | | | | |
| Dicyclohexylamine | | | | | | | |
| 1-methylpiperazine | 0.5 | | | | | | |
| N (2-hydroxyethyl)ethylenediamine | | 0.1 | | | | | |
| 2,2′(ethylenedioxy)bis(ethylamine) | | | 0.1 | | | | |
| Morpholine | | | | 1 | | | |
| Methylbenzylamine | | | | | 1 | | |
| A-methylbenzylamine | | | | | | 1 | |
| Tetraethylenepentamine | | | | | | | 0.5 |
| TFT (min) | 17 | 7 | 11 | 21 | 13 | 38 | 3 |
| Hardness (Shore A) - 2 days | 36/38 | 20/36 | 30/43 | 30/37 | 34/39 | 32/39 | 29/40 |

### Synthesis of TMPTAc-BAm: Michael Addition Reaction

β-Aminoester from TMPTAc and BAm was synthesized in bulk without using catalyst. The molar ratio between the TMPTAc and BAm was maintained at 1:2.7. In a typical experiment, 7.0 gm of TMPTAc was taken in 100 ML round-bottomed (RB) flask. 4.665 gm of BAm was taken in addition funnel and attached to the RB flask. The RB containing TMPTAc was cooled to 10-15° C. using ice-water bath. BAm was added drop wise over 15 minutes under stirring. After two hours, the ice-water bath was removed and the temperature maintained at 20-25° C. The reaction was allowed to proceed for 24 hours under stirring. The product was characterized for their structure using ¹H NMR, 13C NMR and FTIR spectroscopy. The schematic representation of the reaction is shown below.

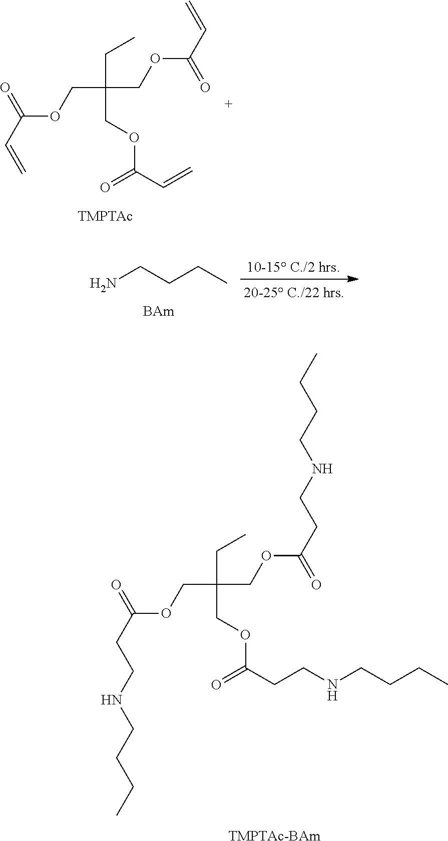

### Additional Amino Ester Compounds

Using similar processes, the following amino esters were produced.

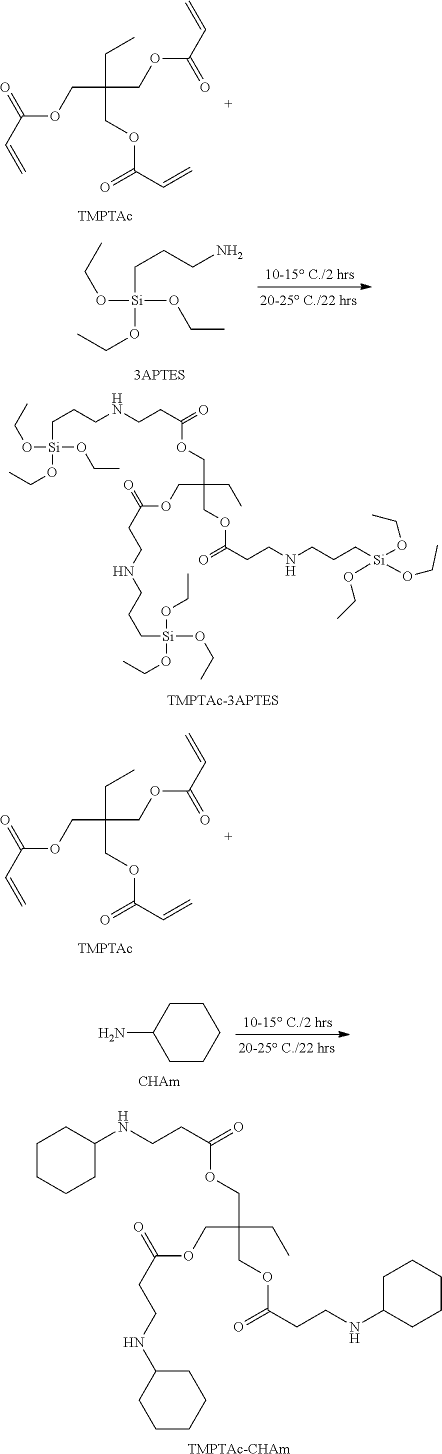

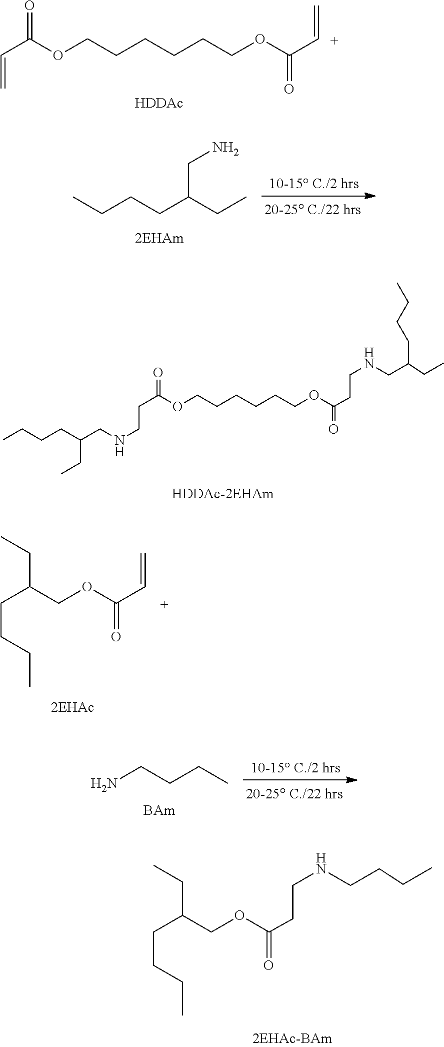

### Examples 1-8

β-Aminoester (TMPTAc-BAm) was tested in a formulation (Component A) comprising silanol, alkoxysilane and fillers. TMPTAc-BAm master batch was prepared using polymer comprising siloxane backbone and polyethylene glycol branches (PEPDMS) at various concentrations (Component B). In a typical experiment, 100 gm of Component A was taken and mixed with 0.5 gm of Component B. The mixed formulation poured in a Teflon mold (L×W×D=50 mm×30 mm×10 mm) and spread evenly using a stainless steel spatula. A 10 gm, stainless steel weight/stainless steel spatula were placed on the surface of formulation to identify the tackiness. Tack free time (TFT) is defined as the time taken for getting a non-tacky surface. The samples were taken out of the mold at the end of 3 days. The hardness (Shore A) was measured both on top and bottom. Other aminoesters were tested without using PEPDMS, The results are summarized in Table 3.

TABLE 3

Component B
(0.5 gm/100 gm
Component A)

| | TMPTAc- | | TFT | Hardness, |
|---|---|---|---|---|
| Example | BAm (gm) | PEPDMS | (min) | Top/Bottom |
| 1 | 0.5 | 0.0 | 63 | 43/43 |
| 2 | 0.4 | 0.1 | 76 | 40/40 |
| 3 | 0.3 | 0.2 | 98 | 38/38 |
| 4 | 0.2 | 0.3 | 124 | 35/36 |

The results show that the formulation comprising β-aminoester as a catalyst provide uniform hardness across the sample and longer TFT. Specific to the mold making application the requirement is longer workability time, when mixed together. Compositions need to show longer TFT at the same time need to have good bulk cure after specified period of time. The above described molecule works very well for such application which is tabulated in Table 4.

TABLE 4

| | 5 | 6 | 7 | 8 |
|---|---|---|---|---|
| Component-A1 | 100 | 100 | 100 | 100 |
| TMPTAc-3APTES | 0.5 | | | |
| TMPTAc-CHAm | | 0.5 | | |
| HDDAc-2EHAm | | | 0.5 | |
| 2EHAc-BAm | | | | 0.5 |

Properties

| | | | | |
|---|---|---|---|---|
| TFT (min) | 92 | 52 | 141 | 90 |
| Hardness (3 day) - top | 31 | 34 | 41 | 43 |
| Hardness (3 day) - bottom | 31 | 34 | 41 | 43 |

### Examples 9-12

The trialkylester (triethyl citrate (TEC)) used in the present study are procured from Aldrich and used as it is without further optimization. The formulations were prepared by mixing the crosslinker, adhesion promoter and catalyst. The formulation details and results are summarized in Table 5. The use of TEC along with the aminosilane adhesion promoters shows good curability which is evident from shore A hardness. The formulation also shows very good adhesion to the substrates such as Al, Glass, ABS. The further adhesion to PBT and AC can be achieved through the mixture of adhesion promoters.

TABLE 5

| | 9 | 10 | 11 | 12 |
|---|---|---|---|---|
| Component A2 | 100 | 100 | 100 | 100 |
| EPS | 1 | 1 | 1 | 1 |
| bis-[gamma-(trimethoxysilyl)propyl]amine | 0.8 | 0.8 | 0.8 | 0.8 |
| N-(beta-aminoethyl)-gamma- | 0.6 | 0.6 | 0.6 | 0.6 |
| aminopropyl trimethoxysilane | | | | |
| gamma-Aminopropyltrimethoxysilane | | | | 1.8 |
| TEC | 0.05 | 0.2 | 0.4 | 0.4 |
| VA-10 | | | | |

Properties

| | | | | |
|---|---|---|---|---|
| TFT (min) | 27 | 32 | 45 | 35 |
| Hardness (3 day) - top | 33 | 35 | 34 | 28 |
| Hardness (3 day) - bottom | 26 | 28 | 27 | 16 |
| Adhesion to Aluminium | ∘ | ∘ | ∘ | ∘ |
| Adhesion to glass | ∘ | ∘ | ∘ | ∘ |
| Adhesion to ABS | ∘ | ∘ | ∘ | ∘ |
| Adhesion to PBT | x | x | x | ∘ |
| Adhesion to AC | x | x | x | ∘ |

### Examples 13-18

Examples 13-18 are prepared as follows using two different aminoester using the component A2, The results indicate that with aminoester it possible to achieve the fast curability and have good adhesion to many different types of substrates the results are shown in Table 6. The results are shown in Table 6:

TABLE 6

| | 13 | 14 | 15 | 16 | 17 | 18 |
|---|---|---|---|---|---|---|
| Component A2 | 100 | 100 | 100 | 100 | 100 | 100 |
| EPS | 2 | 2 | 2 | 2 | 2 | 2 |
| bis-[gamma-(trimethoxysilyl)propyl]amine | 1.2 | | | | | |
| N-(beta-aminoethyl)-gamma-aminopropyl | | 1.2 | | | | |
| trimethoxysilane | | | | | | |
| gamma-Aminopropyltrimethoxysilane | | | 1.2 | | 1.2 | 1.2 |
| HDDAc-2EHAm | 1 | 1 | 1 | | | |
| 2EHAc-Bam | | | | 1 | 1 | 1 |

Properties

| | | | | | | |
|---|---|---|---|---|---|---|
| TFT (min) | 58 | 93 | 43 | 60 | 102 | 50 |
| Hardness (3 day) -top | 38 | 13 | 29 | 39 | 17 | 25 |
| Hardness (3 day) -bottom | 31 | 1 | 9 | 31 | 10 | 8 |
| Adhesion to ABS | NT | ∘ | ∘ | NT | ∘ | ∘ |
| Adhesion to PBT | | ∘ | ∘ | | ∘ | ∘ |
| Adhesion to AC | | ∘ | x | | ∘ | x |

Embodiments of the invention have been described above and modifications and alterations may occur to others upon the reading and understanding of this specification. The claims as follows are intended to include all modifications and alterations insofar as they come within the scope of the claims or the equivalent thereof.

1. A composition for forming a curable polymer composition comprising:
(A) a polymer having at least a reactive silyl group;
(B) a crosslinker or chain extender;
(C) a catalyst comprising an amine compound chosen from a secondary amine, a tertiary amine, a substituted amine, or a combination thereof;
(D) optionally at least one amino-containing adhesion promoter;
(E) optionally a filler component;
(F) optionally a acidic component; and
(G) optionally an auxiliary component comprising an organo-functional silicon compound and or low molecular weight organic polymer and or high boiling solvents.

2. The composition of claim 1, wherein the composition is a two-part composition comprising:
(i) a first portion comprising the polymer (A), optionally the filler component (E), and optionally the acidic compound (F); and
(ii) a second portion comprising the crosslinker (B), the catalyst (C), optionally the adhesion promoter (D), and optionally an organo-functional silicon compound and/or low molecular weight organic polymer or high boiling solvents (G), whereby (i) and (ii) are stored separately until applied for curing by mixing of the components (i) and (ii).

3. The composition of claim 1, wherein the composition is a two-part composition comprising:
(i) a first portion comprising the polymer (A), the crosslinker (B), optionally the filler component (E), and optionally the acidic compound (F); and
(ii) a second portion comprising the catalyst (C), optionally an organo-functional silicon compound and/or low molecular weight organic polymer or high boiling solvents (G), whereby (i) and (ii) are stored separately until applied for curing by mixing of the components (i) and (ii).

4. The composition of claim 1, wherein the catalyst comprises a plurality of amine functional groups.

5. The composition of claim 1, wherein the amine compound comprises one or multiple amine functional group of the formula: 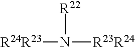
where R²² is independently chosen from hydrogen; a C₁-C₁₅ linear, branched, or cyclic alkyl group; a C₁-C₁₅ linear, branched, or cyclic alkyl group comprising one or more substituents chosen from a halide, N, O or S; a C₆-C₁₀ aryl group; a C₇-C₁₆ linear or branched alkylaryl group; a C₂-C₄ polyalkylene ether; or a linear or branched C₇-C₁₆ heteroaralkyl, heteroalkyl, heterocycloalkyl, or heteroaryl; and
where R²³ and R²⁴ are independently chosen from a C₁-C₁₅ linear, branched, or cyclic alkyl group; a C₁-C₁₅ linear, branched, or cyclic alkyl group comprising one or more substituents chosen from a halide, N, O or S; a C₆-C₁₀ aryl group; a C₇-C₁₆ linear or branched alkylaryl group; a C₂-C₄ polyalkylene ether; a linear or branched C₇-C₁₆ heteroaralkyl; heteroalkyl, heterocycloalkyl, heteroaryl, with the proviso that the N atom is bi-substituted with either of R²³, R²⁴, R²³ and R²⁴, or a combined R²³, R²⁴ in the compound.

6. The composition of claim 1, wherein the catalyst comprises a secondary amine selected from dialkyl and substituted dialkyl amines, dimethylamine, diisopropylamine, dibutylamine, N-methylbutylamine, N,N-diallyl trimethylenediamine, diamylamine, dihexylamine, dioctylamine, N-ethylcetylamine, didodecylamine, ditetradecylamine, diricinoleylamine, N-isopropylstearylamine, N-isoamylhexylamine, N-ethyloctylamine, dioctadecylamine, their homologs and analogs, or a combination of two or more thereof.

7. The composition of claim 1, wherein the catalyst comprises a secondary cycloalkylamine selected from dicyclohexylamine, N-methylcyclohexylamine, dicyclopentylamine, N-octylcyclohexylamine, N-octyl-3,5,5-trimethylcyclohexylamine, diallylamine, N-ethylallylamine, N-octylallylamine, dioleylamine, N-isopropylolelyamine, N-methyl-3,3,5-trimethyl-5-cyclohexenylamine, N-amyl-linoleylamine, N-methyl-propargylamine, diphenylamine, their analogs and homologs, or a combination of two or more thereof.

8. The composition of claim 1, wherein the catalyst comprises a tertiary amine selected from triethylamine, tri-isopropylamine, tributylamine, N-ethyldibutylamine, N-ethyl-N-butylamylamine, N,N-diethyl aniline, triallylamine, N,N-dipropylcyclohexylamine, N,N-dipropyloleyl-amine, trimethylamine, N-octyldiallylamine, N,N-dipropylcyclohexylamine, dimethylaminopropylemine, dimethylaminoethoxypropylamine, pentamethyldiethylylenetriamine, trimethylamine, triethylamine, N-methylmorpholine, N-ethylmorpholine, N,N-dimethylbenzylamine, N,N-dimethylethanolamine, N,N,N′,N′-tetramethyl- 1,4-butanediamine, N,N-dimethylpiperazine, bis(2-dimethylaminoethyl)ether, morpholine, N-substituted morpholines, such as N-methyl or N-ethyl morpholine, 4,4′-(oxydi-2,1-ethanediyl)bis, triethylenediamine, pentamethyl diethylene triamine, dimethyl cyclohexyl amine, N-cetyl N,N-dimethyl amine, N-coco-morpholine, N,N-dimethyl aminomethyl N-methyl ethanol amine, N,N,N′-trimethyl-N′-hydroxyethyl bis(aminoethyl)ether, N,N-bis(3-dimethylaminopropyl)N-isopropanolamine, (N,N-dimethyl)amino-ethoxy ethanol, N,N,N′,N′-tetramethyl hexane diamine, N,N-dimorpholinodiethyl ether, N-methyl imidazole, dimethyl aminopropyl dipropanolamine, bis(dimethylaminopropyl)amino-2-propanol, tetramethylamino bis (propylamine), (dimethyl(aminoethoxyethyl))((dimethyl amine)ethyl)ether, tris(dimethylamino propyl) amine, dicyclohexyl methyl amine, bis(N,N-dimethyl-3-aminopropyl) amine, N,N-bis (3-dimethylaminopropyl)-N-isopropanolamine, 1,3-propanediamine, 1,2-ethylene piperidine, methyl-hydroxyethyl piperazine, dimethylaminopropyl-S-triazine, bisdimethylaminopropylurea, their analogs and homologs, or a combination of two or more thereof.

9. The composition of claim 1, wherein the catalyst comprises a heterocyclic amine selected from piperidine, pyridine, methylpiperazine, 2,2,4,6-tetramethylpiperidine, 2,2,4,6-tetramethyl-tetrahydropyridine, N-ethyl 2,2,4,6 tetramethylpiperidine, 2-aminopyrimidine, 2-aminopyridine, 2-(dimethylamino)pyridine, 4-(dimethylamino)pyridine, 2-hydroxypyridine, imidazole, 2-ethyl-4-methylimidazole, morpholine, N-methylmorpholine, 2-piperidinemethanol, 2-(2-piperidino)ethanol, piperidone, 1,2-dimethyl-1,4,5,6-tetrahydropyrimidine, aziridine, methoymethyldiphenylamine, nicotine, pentobarbital, methadone, cocaine, and triphenylamine, or a combination of two or more thereof.

10. The composition of claim 1, wherein the catalyst comprises a substituted amine is chosen from an amino ester compound.

11. The composition of claim 10, wherein the amino ester compound comprises at least one amino ester functional group.

12. The composition of claim 10, wherein the amino ester compound comprises 1-10 amino ester functional groups.

13. The composition of claim 10, wherein the amino ester compound comprises 1-4 amino ester functional groups.

14. The composition of claim 10, wherein the amino ester compound comprises an amino ester functional group of the formula: 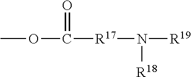
where R¹⁷ is a C₁-C₅ alkyl group, and R¹⁸ and R¹⁹ are independently chosen from hydrogen, a C₁-C₁₀ linear, branched, or cyclic alkyl group, a C₁-C₁₀ linear, branched, or cyclic alkyl group comprising one or more substituents chosen from a halide, N, O or S; a C₆-C₁₀ aryl group; a C₇-C₁₆ alkylaryl group; a C₇-C₁₆ arylalkyl group; a C₂-C₄ polyalkylene ether; a substituted silicon, a substituted siloxane, or a combination of two or more thereof.

15. The composition of claim 10, wherein the amino ester compound is of the formula: 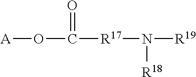
where A is chosen from hydrogen, a C₁-C₁₀ linear, branched, or cyclic alkyl group, a C₁-C₁₀ linear, branched, or cyclic alkyl group comprising one or more substituents chosen from a halide, N, O or S; a C₆-C₁₀ aryl group; a C₇-C₁₆ alkylaryl group; a C₇-C₁₆ arylalkyl group; a C₂-C₄ polyalkylene ether; a substituted silicon, a substituted siloxane, or: 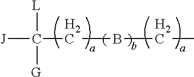
where B is chosen from a C₁-C₁₀ linear, branched, or cyclic alkyl group, a C₁-C₁₀ linear, branched, or cyclic alkyl group comprising one or more substituents chosen from a halide, N, O, or S; a C₆-C₁₀ aryl group; or a silicon compound of the formula
[R¹_{c}R²_{3-c}Si-Z-]ₙ-X-Z-SiR¹_{c}R²_{3-c}
R¹ is chosen from linear or branched alkyl, linear or branched heteroalkyl, cycloalkyl, heterocycloalkyl, aryl, heteroaryl, linear or branched aralkyl, linear or branched heteroaralkyl, or a combination of two or more thereof; Z is chosen from a bond, a divalent linking unit chosen from O, hydrocarbons which optionally contain one or more O, S, or N atom, guanidine-containing, urethane, ether, ester, urea units or a combination of two or more thereof; X is chosen from a polyurethane; a polyester; a polyether; a polycarbonate; a polyolefin; a polyesterether; and a polyorganosiloxane having units of R¹₃SiO_{1/2}, R¹₂SiO, R¹SiO_{3/2}, and/or SiO₂; and G, J, and L are independently chosen from hydrogen an amino ester group of the formula: 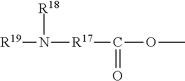
where R¹⁷ is a C₁-C₅ alkyl group, and R¹⁸ and R¹⁹ are independently chosen from hydrogen, a C₁-C₁₀ linear, branched, or cyclic alkyl group, a C₁-C₁₀ linear, branched, or cyclic alkyl group comprising one or more substituents chosen from a halide, N, O or S; a C₆-C₁₀ aryl group; a C₇-C₁₆ alkylaryl group; a C₇-C₁₆ arylalkyl group; a C₂-C₄ polyalkylene ether; a substituted silicon, a substituted siloxane, or a combination of two or more thereof; a is 1 to 10; and b is 0 to 10.

16. The composition of claim 15, wherein B is an alkyl siloxane unit.

17. The composition of claim 10, wherein the amino ester is a poly amino ester comprising a plurality of amino ester units.

18. The composition of claim 10, wherein the amino ester is chosen from a compound of the formula: 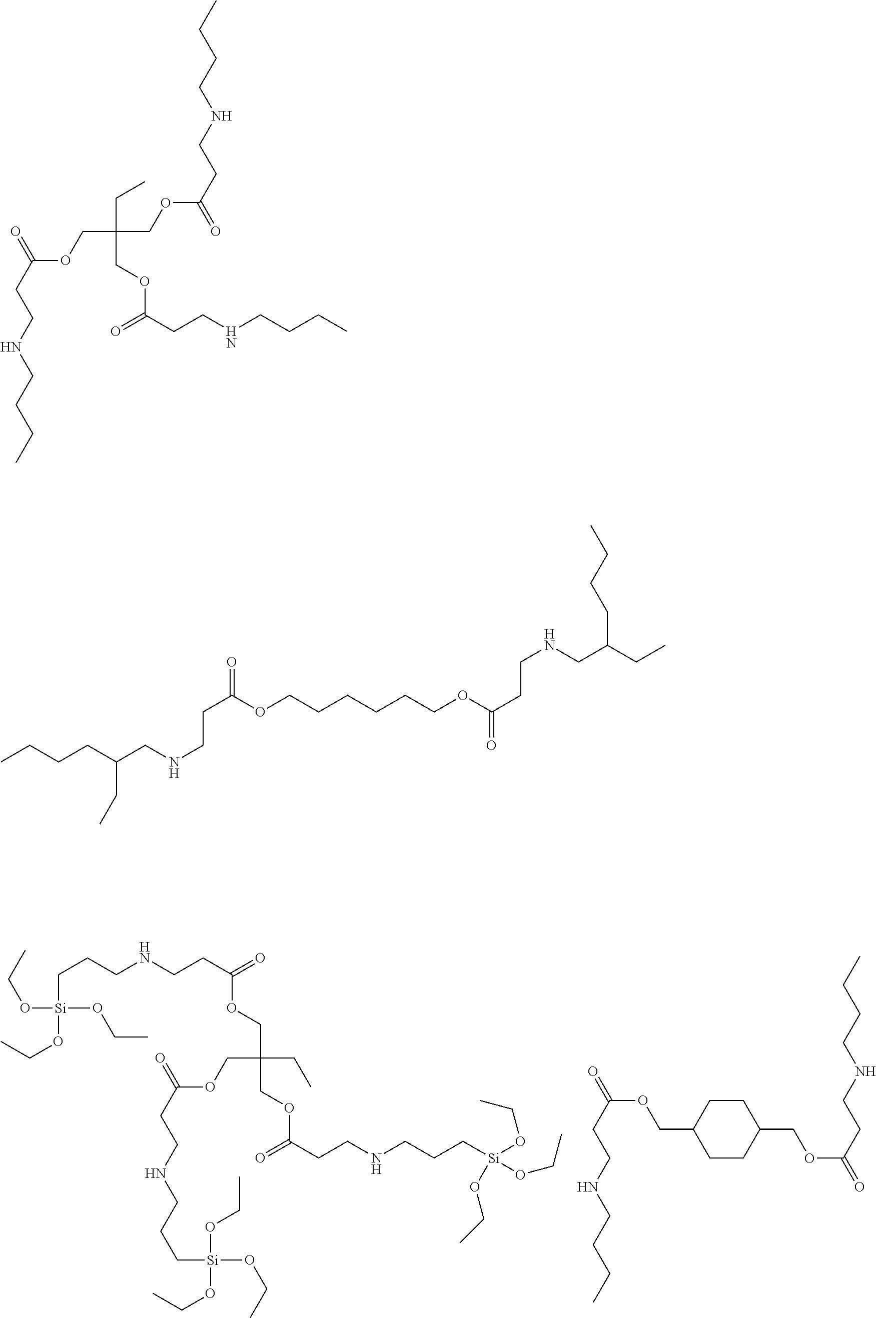 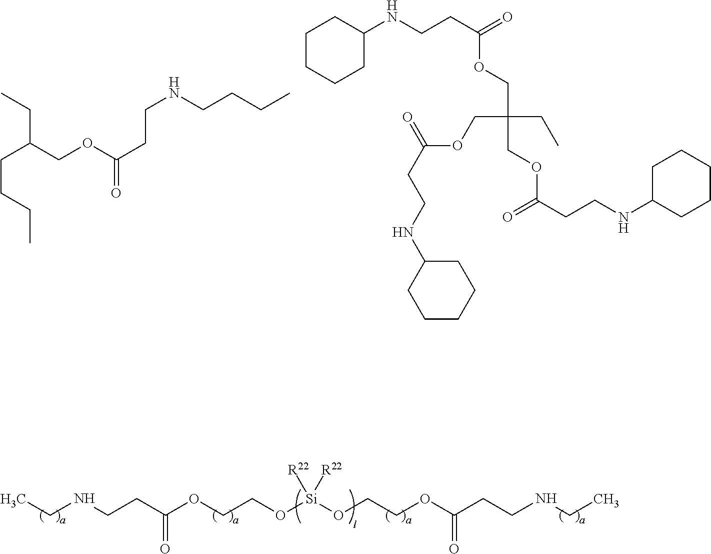
or a combination of two or more thereof.

19. The curable composition of claim 10, wherein the amino ester has a molecular weight of from about 50 g/mol to about 10000 g/mol.

20. The curable composition of claim 10, wherein the amino ester has a pKa of from about 3.0 to about 9.0.

21. The curable composition of claim 10, wherein the catalyst component (C) is present in an amount of from about 0.0001 to about 10.0 wt. pt. based on 100 parts of the polymer component (A).

22. The curable composition of claim 1, wherein the catalyst component (C) is present in an amount of from about 0.15 to about 2.0 wt. pt. based on 100 parts of the polymer component (A).

23. The curable composition of claim 1, wherein the catalyst component (C) is present in an amount of from about 0.5 to about 1.5 wt. pt. of the polymer component (A).

24. The curable composition of claim 1, wherein the adhesion promoter component (D) is present and is chosen from an (aminoalkyl)trialkoxysilane, an (aminoalkyl)alkyldialkoxysilane, a bis(trialkoxysilylalkyl)amine, a tris(trialkoxysilylalkyl)amine, a tris(trialkoxysilylalkyl)cyanuarate, a tris(trialkoxysilylalkyl)isocyanurate, an (epoxyalkyl)trialkoxysilane, an (epoxyalkylether)trialkoxysilane, or a combination of two or more thereof.

25. The curable composition of claim 1 comprising from about 0.1 to about 5 wt. pt. of the adhesion promoter (D) per 100 parts of the polymer (A).

26. The curable composition of claim 1, comprising from about 0.01 to about 5 wt. pt. of the acidic component (F).

27. The curable composition of claim 26, wherein the acidic component (F) is chosen from a carboxylic acid.

28. The curable composition of claim 27, wherein the carboxylic acid is chosen from a C₄-C₃₀ carboxylic acid, a C₄-C₃₀ branched carboxylic acid, or a combination of two or more thereof.

29. The curable composition of claim 1, wherein the filler component is a calcium carbonate filler; a silica filler; fumed or precipitated silicas; ground silicas (Celite™); precipitated and colloidal calcium carbonates; silica gels; hydrophobized silicas; crushed and ground quartz; cristobalite; alumina; aluminum hydroxide; titanium dioxide; zinc oxide; diatomaceous earth; iron oxide; carbon black;
powdered thermoplastics; or clays.

30. A cured polymer formed from the composition of claim 1.

31. The cured polymer of claim 30, wherein the polymer is formed by crosslinking via a condensation reaction and/or a dehydrogenative condensation reaction.

32. A method of making an amino ester comprising reacting a (meth)acrylate with an amine at a temperature of about 20 to about 70° C.

33. The method of claim 32, wherein the reaction comprises (i) adding an amine to an acrylate at a temperature below 30° C. to form a mixture, and (ii) allowing the mixture to react at a temperature of about 20° C. to about 70° C. to form the amino ester.

34. The method of claim 33, wherein the reaction is conducted in the absence of a catalyst.

35. The method of claim 32, wherein the amino ester compound is of the formula: 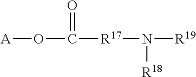
where A is chosen from hydrogen, a C₁-C₁₀ linear, branched, or cyclic alkyl group, a C₁-C₁₀ linear, branched, or cyclic alkyl group comprising one or more substituents chosen from a halide, N, O or S; a C₆-C₁₀ aryl group; a C₇-C₁₆ alkylaryl group; a C₇-C₁₆ arylalkyl group; a C₂-C₄ polyalkylene ether; a substituted silicon, a substituted siloxane, or: 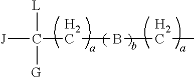
where B is a C₁-C₁₀ linear, branched, or cyclic alkyl group, a C₁-C₁₀ linear, branched, or cyclic alkyl group comprising one or more substituents chosen from a halide, N, O or S; a C₆-C₁₀ aryl group; or a dialkyl siloxane group; and G, J, and L are independently chosen from hydrogen and an amino ester group of the formula: 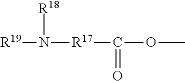
where a is 1 to 10; and b is 0 to 10.
